(19) United States
(12) Patent Application Publication
(10) Pub. No.: US 2025/0120894 A1
(43) Pub. Date: Apr. 17, 2025

(54) ORAL CARE COMPOSITIONS COMPRISING DICARBOXYLIC ACID

(71) Applicant: The Procter & Gamble Company, Cincinnati, OH (US)

(72) Inventors: Andrew Frederic GROTH, Mason, OH (US); Paul Albert SAGEL, Maineville, OH (US); Samuel James ST. JOHN, Cincinnati, OH (US); Ross Strand, Singapore (SG)

(21) Appl. No.: 18/911,401

(22) Filed: Oct. 10, 2024

(30) Foreign Application Priority Data
Oct. 11, 2023 (EP) 23202901.7

(51) Int. Cl. A61K 8/36; A61Q 11/00

(52) U.S. Cl. CPC A61K 8/36; A61Q 11/00

(57) ABSTRACT

Oral care compositions are disclosed comprising a mixture of dentate ligands including mono-, di- and/or tetracarboxylic acid(s) as well as a stannous ion source and a fluoride ions source, such as stannous fluoride. Said oral care compositions show an ability to prevent and/or treat dental erosion and/or caries.

### FIELD OF THE TECHNOLOGY

The present invention is directed to oral care compositions comprising stannous fluoride with monocarboxylic acids, dicarboxylic acids, tricarboxylic acids, tetracarboxylic acids, or combinations thereof and their salts that have an improved hydroxyapatite protective effect towards citric acid challenge or an improved fluoride uptake. The present invention is also directed to methods for treating and/or preventing erosion and/or caries comprising directing a user to apply an oral care composition comprising stannous fluoride with carboxylic acids to an oral cavity.

### BACKGROUND

Oral care compositions, such as toothpaste and/or dentifrice compositions, can be applied to the oral cavity to clean and/or maintain the aesthetics and/or health of the teeth, gums, and/or tongue. Additionally, many oral care compositions are used to deliver active ingredients directly to oral care surfaces. For example, toothpaste compositions can include a stannous ion source. The stannous ion source can deposit on the tooth surfaces creating an acid-resistant coating that helps the teeth resist dissolution from dietary acids. They can also include a fluoride source that helps the teeth resist plaque acids for the prevention of cavities. Stannous fluoride provides both stannous and fluoride ions for the protection of teeth from dietary and plaque acids.

However, stannous fluoride can be challenging to properly formulate in oral care compositions due to reactivity between stannous and other components of oral care compositions. Under-stabilizing or over-stabilizing stannous can lead to lower availability of stannous ions to provide the desired benefit. For example, if the stannous is under-stabilized, the stannous can react with other components of the oral care composition, such as silica, water, etc., which can lead to a lower amount of available stannous ions. Additionally, the remaining under-stabilized stannous, when delivered to the oral cavity, may be hyper-reactive with different oral surfaces, thus impeding the action of other ingredients or causing excess stain. In contrast, if the stannous is over-stabilized or the chelant-stannous interaction is too strong, stannous ions will unavailable when delivered to the oral cavity, which can also lead to a lower amount of bioavailable stannous ions to produce the desired oral care benefit.

Thus, the stannous-chelant ratio and binding affinity must be carefully balanced to maximize the amount of available stannous ions. As such, there is a need for oral care compositions comprising a high amount of available stannous ions that are optimally bioavailable for the desired product benefit.

### SUMMARY

An embodiment of the present invention is directed to an oral care composition comprising:
a) an oral care active, wherein the oral care active comprises a fluoride ion source and a stannous ion source; and
b) a mixture of dentate ligands comprising
   i. a first polydentate ligand, wherein the first polydentate ligand is oxalic acid, a salt thereof, or a combination thereof; and
   ii. a monodentate ligand, wherein the monodentate ligand is a gluconic acid or a salt thereof, or a combination thereof, and the molar ratio of the stannous ions as disclosed in a) to mixture of dentate ligands as disclosed in bi) and bii) is in the range of from 1:3 to 1:4, preferably 1:3 or 1:4; and/or
   iii. a second polydentate ligand, wherein the second polydentate ligand comprises a tridentate ligand or a combination thereof, preferably wherein the second polydentate ligand comprises a tricarboxylic acid, a salt thereof, or a combination thereof.

Another embodiment of the present invention is further directed to an oral care composition comprising:
a) an oral care active, wherein the oral care active comprises a fluoride ion source and a stannous ion source, and
b) a polydentate ligand, wherein the polydentate ligand is a tetracarboxylic acid, preferably wherein the tetracarboxylic acid comprises ethylenediaminetetraacetic acid, ethylenetetracarboxylic acid, ethane-1,1,2,2-tetracarboxylic acid, or a salt thereof or a combination thereof; and
wherein the ratio of the stannous ions to the polydentate ligands is in the range of from about 1:1 to about 1:2, preferably about 1:1.

The present invention, in an embodiment, is further directed to an oral care composition, wherein the pH of the composition is from 4 to 5.

The present invention, in an embodiment, is further directed to the oral care composition as disclosed herein for use in treating erosion, preventing erosion, treating caries, preventing caries, or a combination thereof.

Further, in an embodiment, a method for treating erosion, preventing erosion, treating caries, preventing caries, or a combination thereof is disclosed comprising;
a. depositing the oral care composition as disclosed herein to a suitable application device, wherein the application device may be a toothbrush in one embodiment;
b. applying the oral care composition of a) to the oral cavity surfaces for example by brushing;
c. letting the oral care composition acting on the oral cavity surfaces for at least 2 mins; and
d. removing the oral care composition from the oral cavity by expectorating and optional rinsing.

### DETAILED DESCRIPTION

Embodiments of the present invention is directed to oral care compositions with a stannous-chelant and/or stannous-ligand ratio that results in an optimally bioavailable and shelf-stable composition. Thus, the present invention provides efficacious oral hard tissue solubility reduction benefits and fluoride uptake benefits while simultaneously improving soluble stannous throughout the shelf-life of the oral care composition. Such an achievement was realized with the discovery of optimum ratios of metal ligand ratios that produced the desired stability and reactivity results.

The chelate effect postulates that complexes of polydentate ligands with a metal are more stable than the dentate-normalized equivalent of the monodentate-ligand-stabilized metal complex (e.g., 1 mole of a bidentate ligand in comparison to 2 moles of a similarly structured monodentate ligand) because of a reduction in molar entropy of the bidentate chelate with respect to the monodentate complex.

While not wishing to be bound by theory, in the cases of metals forming complexes in excess ligand and/or in mixed polydentate/monodentate solutions, configurational restrictions in bonding geometries often result when using conventional stabilizers (e.g., citrate anion) that thusly favor the formation of metal-monodentate-polydentate complexes. Consider the case of stannous metal ion being chelated by citrate anion. Sn²⁺ prefers a tetrahedral bonding geometry. The tridentate citrate anion can only occupy two of the four coordinating sites with stannous in this geometry because of steric restrictions. A monodentate ligand (e.g., gluconate) can thus participate in the complex at a third coordination site. The excess electron density (one electron from each of the three coordinating carboxylate anions minus the 2+ formal stannous valency) is then distributed within the Sn bonding orbitals to the fourth coordination site that can acquire a hydrogen-bonded water or hydronium ion when in solution.

While not wishing to be bound by theory, if instead in the previous example, the citrate were replaced by a tetradentate ligand and no monodentate ligand were present that is capable of occupying three coordinating sites simultaneously with a very high binding affinity, the metal chelate could be over-stabilized resulting in a reduction of Sn availability and a loss of oral care benefits. This is a direct result of the chelate effect. Additionally, the metal complex is under-stabilized if too little of the polydentate ligand is used in either the mixed- or polydentate-only cases also resulting in a loss of oral care benefits. Because of the unique properties of stannous ion in solution (tetrahedral bonding geometry with 2+ formal valence) and in the presence of mixed mono/polydentate ligands, Sn²⁺ prefers mixed-dentate complexes. This is because although two polydentate ligands can form a chelate complex, the resulting distribution of electron density is not favored thus providing an enthalpic penalty to formation of the complex.

In the case of monodentate-only stabilized metal complexes, there is no chelate effect and the stabilizing ligands can easily be replaced by chemical moieties with higher binding affinities. This results in under-stabilized stannous in the composition and loss to formula components (e.g., silica) over time. Thus, unexpectedly, an optimum mixture of mono- and polydentate coordinating ligands is needed to properly stabilize the metal ion without impeding its reactivity. As such, the present invention is directed to oral care compositions that provide an unexpectedly high soluble stannous amount throughout the shelf life of the oral care composition while providing optimally reactive stannous capable of providing stannous-related oral care benefits without interfering with the activity of other resulting species.

Finally, didentate ligands are a special case because sometimes they behave as a monodentate ligand and sometimes they behave like a polydentate ligand depending on their size and the orientation/spacing between the carboxylate anions on the central molecule, especially with respect to the coordinating cation. The resulting performance of the metal complexes comprising monodentate, didentate, and/or tridentate ligands varies depending on the behavior of the didentate ligand. As such, if a didentate ligand that behaves like a monodentate ligand is combined with a didentate ligand that behaves like a polydentate ligand, then stannous can be effectively stabilized.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, toothpaste, tooth gel, subgingival gel, emulsion, mouth rinse, mousse, foam, mouth spray, lozenge, chewable tablet, chewing gum, tooth whitening strips, floss and floss coatings, breath freshening dissolvable strips, unit-dose composition, fibrous composition, or denture care or adhesive product. The oral care composition may also be incorporated onto strips or films for direct application or attachment to oral surfaces, such as tooth whitening strips. Examples of emulsion compositions include the emulsions compositions of U.S. Pat. No. 11,147,753, jammed emulsions, such as the jammed oil-in-water emulsions of U.S. Pat. No. 11,096,874. Examples of unit-dose compositions include the unit-dose compositions of U.S. Patent Application Publication No. 2019/0343732.

The term “dentifrice composition”, as used herein, includes tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed. An oral care active for example shows a cosmetic and/or therapeutic benefit for the oral cavity.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients useful in embodiments of the present invention can include the usual and conventional components of mouthwashes or mouth rinses. Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen” or “regimen” can be for the use of two or more separate and distinct treatment steps for oral health, e.g., toothpaste, mouth rinse, floss, toothpicks, spray, water irrigator, massager.

The term “total water content” as used herein means both free water and water that is bound by other ingredients in the oral care composition.

For the purpose of this description, the relevant molecular weight (MW) to be used is that of the material added when preparing the composition, e.g., if the chelant is a citrate species, which can be supplied as citric acid, sodium citrate or indeed other salt forms, the MW used is that of the particular salt or acid added to the composition but ignoring any water of crystallization that may be present.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e., room temperature) unless otherwise specified.

Generally, groups of elements are indicated using the numbering scheme indicated in the version of the periodic table of elements published in Chemical and Engineering News, 63 (5), 27, 1985. In some instances, a group of elements can be indicated using a common name assigned to the group; for example, alkali metals for Group 1 elements, alkaline earth metals for Group 2 elements, and so forth.

Several types of ranges are disclosed in relation to embodiments of the present invention. When a range of any type is disclosed or claimed, the intent is to disclose or claim individually each possible number that such a range could reasonably encompass, including end points of the range as well as any sub-ranges and combinations of sub-ranges encompassed therein.

The oral care composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the dentifrice composition can be incorporated into a film, a strip, a foam, or a fiber-based dentifrice composition.

The oral care composition can include a variety of active and inactive ingredients, such as, for example, but not limited to a hops extract, a dicarboxylic acid, a tin ion source, a calcium ion source, water, a fluoride ion source, zinc ion source, one or more polyphosphates, humectants, surfactants, other ingredients, and the like, as well as any combination thereof, as described below. The section headers below are provided for organization and convenience only. In some cases, a compound can fall within one or more sections. For example, stannous fluoride can be a tin compound and/or a fluoride compound. Additionally, oxalic acid, or salts thereof, can be a dicarboxylic acid, a polydentate ligand, and/or a whitening agent.

### Dentate Ligand

The oral care composition of the present invention comprises a mixture of dentate ligands. The mixture may comprise a monodentate ligand, one or more polydentate ligand(s), or a combination thereof. For example, the mixture may comprise a monodentate ligand and a didentate ligand, or the mixture may comprise a monodentate ligand and a tridentate ligand. In addition, or alternatively, the mixture may comprise for example a first polydentate ligand and a second polydentate ligand, or the mixture may comprise a first polydentate ligand and a monodentate ligand, or the mixture may comprise a first polydentate ligand, a second polydentate ligand and a monodentate ligand. In addition, the first polydentate ligand may be a didentate ligand and the second polydentate ligand may be a didentate ligand, or the first polydentate ligand may be a didentate ligand and the second polydentate ligand may be a tridentate ligand. In addition or alternatively, the oral care composition as disclosed herein may comprise only one polydentate ligand, wherein the polydentate is a tetradentate ligand, such as a tetracarboxylic acid.

### Monodentate Ligand

The oral care composition can comprise monodentate ligand having a molecular weight (MW) of less than 1000 g/mol. A monodentate ligand has a single functional group that can interact with the central atom, such as a tin ion. The monodentate ligand must be suitable for the use in oral care composition, which can be include being listed in Generally Regarded as Safe (GRAS) list with the United States Food and Drug Administration or other suitable list in a jurisdiction of interest.

The monodentate ligand, as described herein, can include a single functional group that can chelate to, associate with, and/or bond to tin. Suitable functional groups that can chelate to, associate with, and/or bond to tin include carbonyl, amine, among other functional groups known to a person of ordinary skill in the art. Suitable carbonyl functional groups can include carboxylic acid, ester, amide, or ketones.

The monodentate ligand can preferably comprise a single carboxylic acid functional group. Suitable monodentate ligands comprising carboxylic acid can include compounds with the formula R—COOH, wherein R is any organic structure. Suitable monodentate ligands comprising carboxylic acid can also include aliphatic carboxylic acid, aromatic carboxylic acid, sugar acid, salts thereof, and/or combinations thereof.

The aliphatic carboxylic acid can comprise a carboxylic acid functional group attached to a linear hydrocarbon chain, a branched hydrocarbon chain, and/or cyclic hydrocarbon molecule. The aliphatic carboxylic acid can be fully saturated or unsaturated and have one or more alkene and/or alkyne functional groups. Other functional groups can be present and bonded to the hydrocarbon chain, including halogenated variants of the hydrocarbon chain. The aliphatic carboxylic acid can also include hydroxyl acids, which are organic compounds with an alcohol functional group in the alpha, beta, or gamma position relative to the carboxylic acid functional group. A suitable alpha hydroxy acid includes lactic acid and/or a salt thereof.

The aromatic carboxylic acid can comprise a carboxylic acid functional group attached to at least one aromatic functional group. Suitable aromatic carboxylic acid groups can include benzoic acid, salicylic acid, and/or combinations thereof.

The carboxylic acid can include formic acid, acetic acid, propionic acid, butyric acid, valeric acid, caproic acid, enanthic acid, caprylic acid, ascorbic acid, benzoic acid, caprylic acid, cholic acid, glycine, alanine, valine, isoleucine, leucine, phenylalanine, linoleic acid, niacin, oleic acid, propanoic acid, sorbic acid, stearic acid, gluconate, lactate, carbonate, chloroacetic acid, dichloroacetic acid, trichloroacetic acid, salts thereof, and/or combinations thereof.

The oral care composition can include from about 1% to about 7.5%, from about 1.5% to about 5%, from about 1.7% to about 4.0%, by weight of the composition, of the monodentate ligand anion.

### Polydentate Ligand

The oral care composition can comprise polydentate ligand having a molecular weight (MW) of less than 1000 g/mol or less than 2500 g/mol. A polydentate ligand has at least two functional groups that can interact with the central atom, such as a tin ion. Additionally, the polydentate ligand must be suitable for the use in oral care composition, which can be include being listed in Generally Regarded as Safe (GRAS) list with the United States Food and Drug Administration or another suitable list in a jurisdiction of interest.

The polydentate ligand, as described herein, can include at least two functional groups that can chelate to, associate with, and/or bond to tin. The polydentate ligand can comprise a bidentate ligand (i.e., with two functional groups), tridentate (i.e., with three functional groups), tetradentate (i.e., with four functional groups), etc.

Suitable functional groups that can chelate to, associate with, and/or bond to tin include carbonyl, phosphate, nitrate, amine, among other functional groups known to a person of ordinary skill in the art. Suitable carbonyl functional groups can include carboxylic acid, ester, amide, or ketones.

The polydentate ligand can comprise two or more carboxylic acid functional groups. Suitable polydentate ligands comprising carboxylic acid can include compounds with the formula HOOC—R—COOH, wherein R is any organic structure. Suitable polydentate ligands comprising two or more carboxylic acid can also include dicarboxylic acid, tricarboxylic acid, tetracarboxylic acid, etc.

The polydentate ligand can comprise oxalic acid, malonic acid, methylmalonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, maleic acid, malic acid, tartaric acid, phthalic acid, citric acid, isocitric acid, aconitic acid, propane-1,2,3-tricarboxylic acid, trimesic acid, ethylenediaminetetraacetic acid, salts thereof, and/or combinations thereof. For example, malonic acid, and oxalic acid, malonic acid and citric acid, oxalic acid and citric acid, or tartaric acid and citric acid, or a combination thereof.

The oral care composition can include from about 1% to about 7.5%, from about 1.5% to about 5%, from about 1.7% to about 4.0%, by weight of the composition, of the polydentate ligand anion.

### Dicarboxylic Acid

The oral care composition comprises dicarboxylic acid. The dicarboxylic acid comprises a compound with two carboxylic acid functional groups. The dicarboxylic acid can comprise a compound or salt thereof defined by Formula VIII-A, Formula VIII-B, and/or Formula VIII-C.

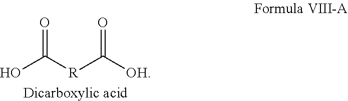

R can be null, alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

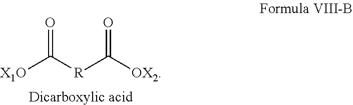

R can be null, alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

X₁ and X₂ can independently be H, alkali metal, alkali earth metal, transition metal, or combinations thereof. Suitable alkali metals include lithium, sodium, potassium, or combinations thereof. Suitable alkali earth metals include magnesium, calcium, barium, or combinations thereof. Suitable transitional metals include titanium, chromium, iron, nickel, copper, zinc, tin, gold, silver, or combinations thereof.

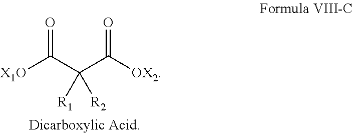

R₁ can be null, alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

X₁ and X₂ can independently be H, alkali metal, alkali earth metal, transition metal, or combinations thereof. Suitable alkali metals include lithium, sodium, potassium, or combinations thereof. Suitable alkali earth metals include magnesium, calcium, barium, or combinations thereof. Suitable transitional metals include titanium, chromium, iron, nickel, copper, zinc, tin, gold, silver, or combinations thereof.

The dicarboxylic acid can be added to a formulation as a neutral acid (as shown in Formula VIII-A) or as a dicarboxylate monosalt (where one of the carboxylic acid functional groups is a salt and the other is neutral), a dicarboxylate disalt (where both of the carboxylic acid functional groups are salts), or combinations thereof. Additionally, as is well known to a person of ordinary skill in the art, whether or not that one or both of the carboxylic acid functional groups of the dicarboxylic acid are neutral or charged in solution, can be influenced by the pH of the solution. For example, a neutral dicarboxylic acid can be added to an aqueous solution and one or two protons from the two carboxylic acid functional groups can be removed if the pH is lower than the pKa of the carboxylic acid functional group, as shown below in Formula VIII-D.

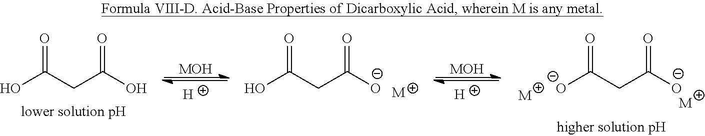

The dicarboxylic acid can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, maleic acid, tartaric acid, phthalic acid, methylmalonic acid, dimethylmalonic acid, tartaric acid, tartronic acid, mesoxalic acid, dihydroxymalonic acid, dihydroxymalonic acid, fumaric acid, terephthalic acid, glutaric acid, salts thereof, or combinations thereof. The dicarboxylic acid can comprise suitable salts of dicarboxylic acid, such as, for example, when the dicarboxylic acid includes a salt of oxalic acid: monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The dicarboxylic acid can also include hydrates of the dicarboxylic acid and/or a hydrate of a salt of the dicarboxylic acid.

Suitable dicarboxylic acid compounds include malonic acid, methylmalonic acid, tartronic acid, malic acid, maleic acid, dimethylmalonic acid, mesoxalic acid, dihydroxymalonic acid, oxalic acid, tartaric acid, salts thereof, or combinations thereof. These dicarboxylic acid compounds are particularly suitable as these compounds have been shown to have an unexpectedly high whitening benefit. While not wishing to be bound by theory, it is believed that particular dicarboxylic acid compounds have an unexpectedly high affinity to certain cationic crosslinking agents typically found in the colored matrix on the oral hard tissue surfaces, thereby resulting in the removal of stain from the surface. Without being bound by theory, it is hypothesized that the whitening efficacy of the dicarboxylics acids and their corresponding anions is driven by the ability of the dicarboxylic acid to reach and remove cationic bridges between chromophores and the tooth surface as well as chromophores and the pellicle proteins.

Suitable dicarboxylic acid compounds include dicarboxylic acids described by Formula VIII-A, wherein R is null, comprises a methylene or ethylene with one or two substitutions, and/or an acetyl group.

### Fluoride

The oral care composition comprises fluoride, which can be provided by a fluoride ion source. The fluoride ion source can comprise one or more fluoride containing compounds, such as stannous fluoride, sodium fluoride, titanium fluoride, calcium fluoride, calcium phosphate silicate fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of, or free of stannous fluoride. Thus, the oral) care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 5%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, be substantially free of, or be free of a fluoride ion source.

### Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, K, Cu, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of metal and/or a metal ion source.

### Tin

An oral care composition according to embodiments of the present invention comprise tin, which can be provided by a tin ion source. The tin ion source can be any suitable compound that can provide tin ions in an oral care composition and/or deliver tin ions to the oral cavity when the oral care composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 5%, from about 0.2% to about 1%, from about 0.4% to about 1%, or from about 0.3% to about 0.6%, by weight of the oral care composition, of tin and/or a tin ion source.

### Zinc

The oral care composition can comprise zinc, which can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc and/or zinc ion source may be present in the total oral care composition at an amount of from about 0.01% to about 10%, from about 0.2% to about 1%, from about 0.4% to about 1%, from about 0.5% to about 1.5%, or from about 0.3% to about 0.6%, by weight of the oral care composition. Alternatively, the oral care composition can be essentially free of, substantially free of, or free of zinc.

### Potassium

The oral care composition can comprise potassium, which can be provided by a potassium ion source. The potassium ion source can comprise one or more potassium containing compounds, such as potassium nitrate, potassium fluoride, potassium chloride, or combinations thereof.

The oral care composition can comprise from about 0.01% to about 10%, from about 0.2% to about 1%, from about 0.4% to about 1%, or from about 0.3% to about 0.6%, by weight of the oral care composition, of potassium and/or potassium ion source. Alternatively, the oral care composition can be essentially free of, substantially free of, or free of potassium.

pH

The pH of the oral care compositions as described herein can be from about 4 to about 6, from about 4.5 to about 5.5, from about 4 to less than 5.5, from about 4.5 to less than 5.5, greater than 4 to less than 5, greater than 4 to about 4.9, from about 4.9, from about 4 to about 5.4, from about 4 to about 5.3, from about 4 to about 5.2, from about 4 to about 5.1, from about 4 to about 5, from about 4 to about 4.9, from about 4 to about 4.8, from about 4 to about 4.7. The pH of a mouth rinse solution can be determined as the pH of the neat solution. The pH of a dentifrice composition can be determined as a slurry pH, which is the pH of a mixture of the dentifrice composition and water, such as a 1:4, 1:3, or 1:2 mixture of the dentifrice composition and water.

As the oral care composition comprises one or more dicarboxylic acids, a preferred pH is below about 7 or below about 6 due to the pKa of the dicarboxylic acid. While not wishing to be bound by theory, it is believed that the dicarboxylic acid displays unique behavior when the pH is below about 7, in particular below about 6, but surfaces in the oral cavity can also be sensitive to a low pH. Additionally, at pH values above about pH 7, the metal ion source can react with water and/or hydroxide ions to form insoluble metal oxides and/or metal hydroxides. The formation of these insoluble compounds can limit the ability of dicarboxylates to stabilize metal ions in oral care compositions and/or can limit the interaction of dicarboxylates with target metal ions in the oral cavity. Additionally, at pH values less than 4, the potential for demineralization is greatly increased. Consequently, the oral care compositions comprising dicarboxylic acid, as described herein, can preferably have a pH from about 4 to about 6, from about 4 to about 5, from about 4 to less than 5, from about 4 to about 4.9, or from about 4.5 to less than 5.5 to minimize metal hydroxide/metal oxide formation and any increased demineralization in the oral cavity.

The pH of the oral care composition, as described herein, can be measured either immediately upon mixing, or upon aging the composition by placing the oral care composition at ambient or accelerated temperature and humidity conditions, such as including measuring the pH at a temperature of 25° C., 30° C. and/or 40° C. with a 30%, 60% and/or 75% relative humidity for about 28 days or longer prior to measuring the pH.

### Buffering Agents

The oral care composition can comprise one or more buffering agents. Buffering agents, as used herein, refer to agents that can be used to adjust the slurry pH of the oral care compositions. The buffering agents include alkali metal hydroxides, carbonates, sesquicarbonates, borates, silicates, phosphates, imidazole, and mixtures thereof. Specific buffering agents include monosodium phosphate, trisodium phosphate, sodium hydroxide, potassium hydroxide, alkali metal carbonate salts, sodium carbonate, imidazole, pyrophosphate salts, citric acid, and sodium citrate. The oral care composition can comprise one or more buffering agents each at a level of from about 0.1% to about 30%, from about 1% to about 10%, or from about 1.5% to about 3%, by weight of the present composition.

### Orally Acceptable Carrier

The oral care composition as disclosed herein may comprise an orally acceptable carrier in addition to the oral care actives and the components specified in detail. An orally acceptable carrier may comprise surfactant(s), thickening agent(s), abrasive(s), humectant(s), water and other ingredient(s).

### Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants.

Suitable anionic surfactants include, for example, the water soluble salts of alkyl sulfates having from 8 to 20 carbon atoms in the alkyl radical and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Other suitable anionic surfactants include sarcosinates, such as sodium lauroyl sarcosinate, taurates, sodium lauryl sulfoacetate, sodium lauroyl isethionate, sodium laureth carboxylate, and sodium dodecyl benzene sulfonate. Combinations of anionic surfactants can also be employed.

Zwitterionic or amphoteric surfactants useful herein include derivatives of aliphatic quaternary ammonium, phosphonium, and Sulfonium compounds, in which the aliphatic radicals can be straight chain or branched, and one of the aliphatic substituents contains from 8 to 18 carbon atoms and one contains an anionic water-solubilizing group, e.g., carboxy, sulfonate, sulfate, phosphate or phosphonate. Suitable betaine surfactants are disclosed in U.S. Pat. No. 5,180,577. Typical alkyl dimethyl betaines include decyl betaine or 2-(N-decyl-N,N-dimethylammonio) acetate, coco-betaine or 2-(N-coco-N,N-dimethyl ammonio)acetate, myristyl betaine, palmityl betaine, lauryl betaine, cetyl betaine, cetyl betaine, stearyl betaine, etc. The amidobetaines can be exemplified by cocoamidoethyl betaine, cocoamidopropyl betaine (CADB), and lauramidopropyl betaine. Other suitable amphoteric surfactants include betaines, sultaines, sodium laurylamphoacetates, alkylamphodiacetates, and/or combinations thereof.

Suitable cationic surfactants include, for example, derivatives of quaternary ammonium compounds having one long alkyl chain containing from 8 to 18 carbon atoms such as lauryl trimethylammonium chloride; cetyl pyridinium chloride; cetyl trimethyl-ammonium bromide; cetyl pyridinium fluoride or combinations thereof.

Suitable nonionic surfactants include, for example, compounds produced by the condensation of alkylene oxide groups (hydrophilic in nature) with an organic hydrophobic compound which may be aliphatic or alkylaromatic in nature. Examples of suitable nonionic surfactants can include the Pluronics® which are poloxamers, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and combinations of such materials. Other suitable non-ionic surfactants includes alkyl glucamides, alkyl glucosides, and/or combinations thereof.

The oral care composition can comprise one or more surfactants each at a level from about 0.01% to about 15%, from about 0.3% to about 10%, or from about 0.3% to about 2.5%, by weight of the oral care composition

### Thickening Agent

The oral care composition can comprise one or more thickening agents. Thickening agents can be useful in the oral care compositions to provide a gelatinous structure that stabilizes the composition against phase separation. Suitable thickening agents include polysaccharides, polymers, and/or silica thickeners.

The thickening agent can comprise one or more polysaccharides. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, microcrystalline cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

Other polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. Alternatively, the oral care composition can be free of, essentially free of, or substantially free of a copolymer of maleic anhydride and methyl vinyl ether. The polymer can comprise polyacrylate crosspolymer, such as polyacrylate crosspolymer-6. Suitable sources of polyacrylate crosspolymer-6 can include Sepimax Zen™ commercially available from Seppic.

The thickening agent can comprise inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can comprise from 0.01% to about 15%, from 0.1% to about 10%, from about 0.2% to about 5%, or from about 0.5% to about 2% of one or more thickening agents.

### Abrasive

The oral care composition of embodiments of the present invention can comprise an abrasive. Abrasives can be added to oral care formulations to help remove surface stains from teeth. The oral care can include a calcium abrasive and/or a non-calcium abrasive, such as a silica abrasive.

The oral care composition can comprise a calcium abrasive. The calcium abrasive can be any suitable abrasive compound that can provide calcium ions in an oral care composition and/or deliver calcium ions to the oral cavity when the oral care composition is applied to the oral cavity. The oral care composition can comprise from about 5% to about 70%, from about 10% to about 60%, from about 20% to about 50%, from about 25% to about 40%, or from about 1% to about 50% of a calcium abrasive. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The oral care composition can comprise a non-calcium abrasive such as bentonite, silica gel (by itself, and of any structure), precipitated silica, amorphous precipitated silica (by itself, and of any structure as well), hydrated silica, perlite, titanium dioxide, calcium pyrophosphate, dicalcium phosphate dihydrate, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. Such materials can be introduced into the oral care compositions to tailor the polishing characteristics of the target dentifrice formulation. The oral care composition can comprise from about 5% to about 70%, from about 10% to about 50%, from about 10% to about 60%, from about 20% to about 50%, from about 25% to about 40%, or from about 1% to about 50%, by weight of the oral care composition, of the non-calcium abrasive.

Alternatively, the oral care composition can be essentially free of, substantially free of, essentially free of, or free of silica, alumina, or any other non-calcium abrasive. The oral care composition can comprise less than about 5%, less than about 1%, less than about 0.5%, less than about 0.1%, or 0% of a non-calcium abrasive, such as silica and/or alumina.

The oral care composition can also comprise a silica abrasive, such as silica gel (by itself, and of any structure), precipitated silica, amorphous precipitated silica (by itself, and of any structure as well), hydrated silica, and/or combinations thereof. The oral care composition can comprise from about 5% to about 70%, from about 10% to about 60%, from about 10% to about 50%, from about 20% to about 50%, from about 25% to about 40%, or from about 1% to about 50% of a silica abrasive.

Where the oral care composition comprises a dicarboxylic acid, the oral care composition can include a low level of or no abrasive as the dicarboxylic acid can provide a high enough whitening benefit that an abrasive is not necessary.

While mouth rinse compositions typically do not include abrasive, dentifrice compositions typically do include abrasive. However, the dentifrice compositions and/or toothpaste compositions of embodiments of the present invention can include a low level of or no abrasive. As such, the oral care composition or dentifrice composition can comprise less than about 5%, from about 0.5% to about 2%, or less than about 2%, by weight of the composition, of abrasive. The oral care composition or dentifrice composition can also be essentially free of, substantially free of, or free of abrasive.

### Humectant

The oral care composition can comprise one or more humectants, or have low levels of a humectant, or be essentially free of, be substantially free of, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Water

The oral care composition according to embodiments of the present invention can be anhydrous, a low water formulation, or a high water formulation. In total, the oral care composition can comprise from 0% to about 99%, from about 5% to about 75%, about 20% or greater, about 30% or greater, about 50% or greater, up to about 45%, or up to about 75%, by weight of the composition, of water.

In a high water oral care composition and/or toothpaste formulation, the oral care composition comprises from about 45% to about 75%, by weight of the composition, of water. The high water oral care composition and/or toothpaste formulation can comprise from about 45% to about 65%, from about 45% to about 55%, or from about 46% to about 54%, by weight of the composition, of water. The water may be added to the high water formulation and/or may come into the composition from the inclusion of other ingredients.

In a low water oral care composition and/or toothpaste formulation, the oral care composition comprises from about 5% to about 45%, by weight of the composition, of water. The low water oral care composition can comprise from about 5% to about 35%, from about 10% to about 25%, or from about 20% to about 25%, by weight of the composition, of water. The water may be added to the low water formulation and/or may come into the composition from the inclusion of other ingredients.

In an anhydrous oral care composition and/or toothpaste formulation, the oral care composition comprises less than about 10%, by weight of the composition, of water. The anhydrous composition comprises less than about 5%, less than about 1%, or 0%, by weight of the composition, of water. The water may be added to the anhydrous formulation and/or may come into the composition from the inclusion of other ingredients.

The oral care composition can also be a mouth rinse formulation. A mouth rinse formulation can comprise from about 75% to about 99%, from about 75% to about 95%, or from about 80% to about 95% of water.

The dentifrice composition can also comprise other orally acceptable carrier materials, such as alcohol, humectants, polymers, surfactants, and acceptance improving agents, such as flavoring, sweetening, coloring and/or cooling agents.

### Antibacterial Agents

The oral care composition can comprise one or more antibacterial agents. Suitable antibacterial agents include any molecule that provides antibacterial activity in the oral cavity. Suitable antibacterial agents include hops acids, tin ion sources, benzyl alcohol, sodium benzoate, menthylglycyl acetate, menthyl lactate, L-menthol, o-neomenthol, chlorophyllin copper complex, phenol, oxyquinoline, and/or combinations thereof.

The oral care composition can comprise from about 0.01% to about 10%, from about 1% to about 5%, or from about 0.5% to about 15% of an antibacterial agent.

### Bioactive Materials

The oral care composition can also include bioactive materials suitable for the remineralization of a tooth. Suitable bioactive materials include bioactive glasses, Novamin™, Recaldent™, hydroxyapatite, one or more amino acids, such as, for example, arginine, citrulline, glycine, lysine, or histidine, or combinations thereof. Suitable examples of compositions comprising arginine are found in U.S. Pat. Nos. 4,154,813 and 5,762,911, which are herein incorporated by reference in their entirety. Other suitable bioactive materials include any calcium phosphate compound. Other suitable bioactive materials include compounds comprising a calcium source and a phosphate source.

Amino acids are organic compounds that contain an amine functional group, a carboxyl functional group, and a side chain specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutonic acid, diaminoproprionic acid, salts thereof, and/or combinations thereof.

Bioactive glasses are comprising calcium and/or phosphate which can be present in a proportion that is similar to hydroxyapatite. These glasses can bond to the tissue and are biocompatible. Bioactive glasses can include a phosphopeptide, a calcium source, phosphate source, a silica source, a sodium source, and/or combinations thereof.

The oral care composition can comprise from about 0.01% to about 20%, from about 0.1% to about 10%, or from about 1% to about 10% of a bioactive material by weight of the oral care composition.

### Quaternary Ammonium Compound

The oral care composition can include quaternary ammonium compound. The quaternary ammonium compounds in the compositions of embodiments of the present invention can include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Cetylpyridinium chloride, cetyl pyridinium fluoride, tetradecylpyridinium chloride, N-tetradecyl-4-ethyl pyridinium chloride, domiphen bromide, benzalkonium chloride, benzethonium chloride, methyl benzethonium chloride, dodecyl trimethyl ammonium bromide, dodecyl dimethyl (2-phenoxyethyl) ammonium bromide, benzyl dimethoxystearyl ammonium chloride, quaternized 5-amino-1,3-bis(2-ethyl-hexyl)-5-methyl hexa hydropyrimidine, lauryl trimethylammonium chloride, cocoalkyl trimethylammonium chloride, cetyl trimethylammonium bromide, di-isobutylphenoxyethyl-dimethylbenzylammonium chloride, dodecyl trimethyl ammonium bromide, are exemplary of typical quaternary ammonium antimicrobial agents. Other compounds are bis [4-(R-amino)-1-pyridinium] alkanes as disclosed in U.S. Pat. No. 4,206,215 to Bailey. The pyridinium compounds are the preferred quaternary ammonium compounds, particularly preferred being cetylpyridinium, or tetradecylpyridinium halide salts (i.e., chloride, bromide, fluoride and iodide). Particularly preferred are cetylpyridinium chloride and fluoride salts.

The oral care composition can comprise at least about 0.025%, at least about 0.035%, at least about 0.045% to about 1.0%, from about 0.025% to about 1%, or from about 0.01% to about 10%, by weight of the composition, of the quaternary ammonium compound. Alternatively, the oral care composition can be essentially free of, substantially free of, or free of a quaternary ammonium compound.

### Prenylated Flavonoids

The oral care composition can comprise prenylated flavonoid. Flavonoids are a group of natural substances found in a wide range of fruits, vegetables, grains, bark, roots, stems, flowers, tea, and wine. Flavonoids can have a variety of beneficial effects on health, such as antioxidative, anti-inflammatory, antimutagenic, anticarcinogenic, and antibacterial benefits. Prenylated flavonoids are flavonoids that include at least one prenyl functional group (3-methylbut-2-en-1-yl, as shown in Formula IX), which has been previously identified to facilitate attachment to cell membranes. Thus, while not wishing to being bound by theory, it is believed that the addition of a prenyl group, i.e. prenylation, to a flavonoid can increase the activity of the original flavonoid by increasing the lipophilicity of the parent molecule and improving the penetration of the prenylated molecule into the bacterial cell membrane. Increasing the lipophilicity to increase penetration into the cell membrane can be a double-edged sword because the prenylated flavonoid will tend towards insolubility at high Log P values (high lipophilicity). Log P can be an important indicator of antibacterial efficacy.

As such, the term prenylated flavonoids can include flavonoids found naturally with one or more prenyl functional groups, flavonoids with a synthetically added prenyl functional group, and/or prenylated flavonoids with additional prenyl functional groups synthetically added.

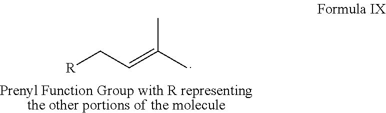

Other suitable functionalities of the parent molecule that improve the structure-activity relationship (e.g,. structure-MIC relationship) of the prenylated molecule include additional heterocycles containing nitrogen or oxygen, alkylamino chains, or alkyl chains substituted onto one or more of the aromatic rings of the parent flavonoid.

Flavonoids can have a 15-carbon skeleton with at least two phenyl rings and at least one heterocyclic ring. Some suitable flavonoid backbones can be shown in Formula X (flavone backbone), Formula XI (isoflavan backbone), and/or Formula XII (neoflavonoid backbone).

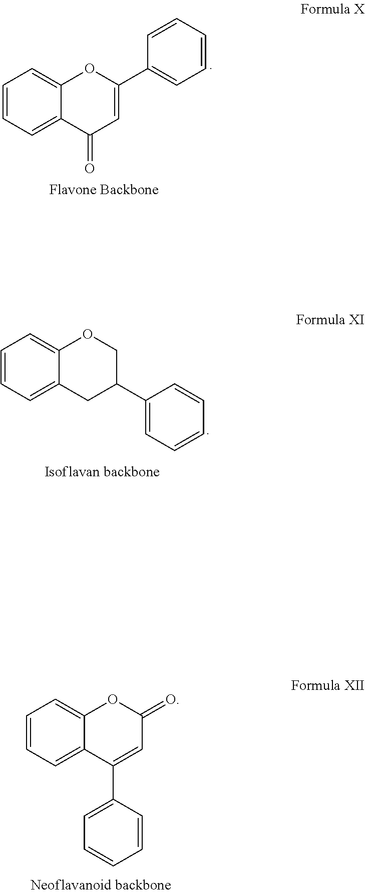

Other suitable subgroups of flavonoids include anthocyanidins, anthoxanthins, flavanones, flavanonols, flavans, isoflavonoids, chalcones and/or combinations thereof.

Prenylated flavonoids can include naturally isolated prenylated flavonoids or naturally isolated flavonoids that are synthetically altered to add one or more prenyl functional groups through a variety of synthetic processes that would be known to a person of ordinary skill in the art of synthetic organic chemistry.

Other suitable prenylated flavonoids can include Bavachalcone, Bavachin, Bavachinin, Corylifol A, Epimedin A, Epimedin A1, Epimedin B, Epimedin C, Icariin, Icariside I, Icariside II, Icaritin, Isobavachalcone, Isoxanthohumol, Neobavaisoflavone, 6-Prenylnaringenin, 8-Prenylnaringenin, Sophoraflavanone G, (−)-Sophoranone, Xanthohumol, Quercetin, Macelignan, Kuraridin, Kurarinone, Kuwanon G, Kuwanon C, Panduratin A, 6-geranylnaringenin, Australone A, 6,8-Diprenyleriodictyol, dorsmanin C, dorsmanin F, 8-Prenylkaempferol, 7-O-Methylluteone, luteone, 6-prenylgenistein, isowightcone, lupiwighteone, and/or combinations thereof. Other suitable prenylated flavonoids include cannflavins, such as Cannflavin A, Cannflavin B, and/or Cannflavin C.

Preferably, the prenylated flavonoid has a high probability of having a MIC of less than about 25 ppm for S. aureus, a gram-positive bacterium. Suitable prenylated flavonoids include Bavachin, Bavachinin, Corylifol A, Icaritin, Isoxanthohumol, Neobavaisoflavone, 6-Prenylnaringenin, 8-Prenylnaringenin, Sophoraflavanone G, (−)-Sophoranone, Kurarinone, Kuwanon C, Panduratin A, and/or combinations thereof.

Preferably, the prenylated flavonoid has a high probability of having a MIC of less than about 25 ppm for E. coli, a gram-negative bacterium. Suitable prenylated flavonoids include Bavachinin, Isoxanthohumol, 8-Prenylnaringenin, Sophoraflavanone G, Kurarinone, Panduratin A, and/or combinations thereof.

Approximately 1000 prenylated flavonoids have been identified from plants. According to the number of prenylated flavonoids reported before, prenylated flavonones are the most common subclass and prenylated flavanols is the rarest sub-class. Even though natural prenylated flavonoids have been detected to have diversely structural characteristics, they have a narrow distribution in plants, which are different to the parent flavonoids as they are present almost in all plants. Most of prenylated flavonoids are found in the following families, including Cannabaceae, Guttiferae, Leguminosae, Moraceae, Rutaceae and Umbelliferae. Leguminosas and Moraceae, due to their consumption as fruits and vegetables, are the most frequently investigated families and many novel prenylated flavonoids have been explored. Humulus lupulus of the Cannabaceae include 8-prenylnaringenin and xanthohumol, which can play a role in the health benefits of beer.

The prenylated flavonoid can be incorporated through a hops extract, incorporated in a separately added extract, or added as a separate component of the oral care compositions disclosed herein.

Suitable prenylated flavonoids can have a particular octanol-water partitioning coefficient. The octanol-water partitioning coefficient can be used to predict the lipophilicity of a compound. Without wishing to being bound by theory, it is believed that compounds that fall within the ranges described herein will be able to enter and/or disrupt the primarily hydrophobic phospholipid bilayer that makes of the cell membrane of microorganisms. Thus, the octanol-water partitioning coefficient can be correlated to the antibacterial effect of prenylated flavonoids. Suitable prenylated flavonoids can have a log P of at least about 2, at least about 4, from about 2 to about 10, from about 4 to about 10, from about 4 to about 7, or from about 4 to about 7.

The oral care composition can comprise at least about 0.001%, from about 0.001% to about 5%, from about 0.01% to about 2%, from about 0.0001% to about 2%, or at least about 0.05% of prenylated flavonoid.

### Amino Acid

The oral care composition can comprise amino acid. The amino acid can comprise one or more amino acids, peptide, and/or polypeptide, as described herein.

Amino acids, as in Formula XIII, are organic compounds that contain an amine functional group, a carboxyl functional group, and a side chain (R in Formula XIII) specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, and/or combinations thereof.

Suitable amino acids include the compounds described by Formula XIII, either naturally occurring or synthetically derived. The amino acid can be zwitterionic, neutral, positively charged, or negatively charged based on the R group and the environment. The charge of the amino acid, and whether particular functional groups, can interact with tin at particular pH conditions, would be well known to one of ordinary skill in the art.

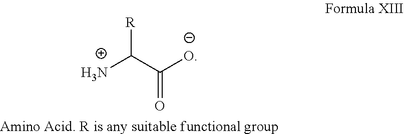

Suitable amino acids include one or more basic amino acids, one or more acidic amino acids, one or more neutral amino acids, or combinations thereof.

The oral care composition can comprise from about 0.01% to about 20%, from about 0.1% to about 10%, from about 0.5% to about 6%, or from about 1% to about 10% of amino acid, by weight of the oral care composition.

The term “neutral amino acids” as used herein include not only naturally occurring neutral amino acids, such as alanine, asparagine, cysteine, glutamine, glycine, isoleucine, leucine, methionine, phenylalanine, proline, serine, threonine, tryptophan, tyrosine, valine, but also biologically acceptable amino acids which have an isoelectric point in range of pH 5.0 to 7.0. The biologically preferred acceptable neutral amino acid has a single amino group and carboxyl group in the molecule or a functional derivative hereof, such as functional derivatives having an altered side chain albeit similar or substantially similar physio chemical properties. In a further embodiment the amino acid would be at minimum partially water soluble and provide a pH of less than 7 in an aqueous solution of 1 g/1000 ml at 25° C.

Accordingly, neutral amino acids suitable for use in embodiments of the present invention include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, or mixtures thereof. Preferably, the neutral amino acids used in embodiments of the present invention may include asparagine, glutamine, glycine, salts thereof, or mixtures thereof. The neutral amino acids may have an isoelectric point of 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0, in an aqueous solution at 25° C. Preferably, the neutral amino acid is selected from proline, glutamine, or glycine, more preferably in its free form (i.e., uncomplexed). If the neutral amino acid is in its salt form, suitable salts include salts known in the art to be pharmaceutically acceptable salts considered to be physiologically acceptable in the amounts and concentrations provided. Preferably the neutral amino acid is present in the amount of from about 0.0001% to about 10%, preferably from about 0.05% to about 5%, preferably from about 0.1% to about 3%, preferably from about 0.5% to about 3%, preferably from about 1% to about 3%, by weight of the composition. In one aspect, the neutral amino acid is glutamine (or salt thereof). In another aspect, the neutral amino acid is proline (or salt thereof). In yet another aspect, the neutral amino acid is glycine (or salt thereof).

The oral care composition can comprise from about 0.0001% to about 20%, from about 0.1% to about 10%, from about 0.5% to about 6%, or from about 1% to about 10% of neutral amino acid, by weight of the oral care composition.

Humulus lupulus

Oral care compositions of the present invention can comprise hops. The hops can comprise at least one hops compound from Formula I and/or Formula IV. The compound from Formula I and/or Formula IV can be provided by any suitable source, such as an extract from Humulus lupulus or Hops, Humulus lupulus itself, a synthetically derived compound, and/or salts, prodrugs, or other analogs thereof. The hops extract can comprise one or more hops alpha acids, one or more hops iso-alpha acids, one or more hops beta acids, one or more hops oils, one or more flavonoids, one or more solvents, and/or water. Suitable hops alpha acids (generically shown in Formula I) can include humulone (Formula II), adhumulone, cohumulone, posthumulone, prehumulone, and/or mixtures thereof. Suitable hops iso-alpha acids can include cis-isohumulone and/or trans-isohumulone. The isomerization of humulone into trans-isohumulone can be represented by Formula III.

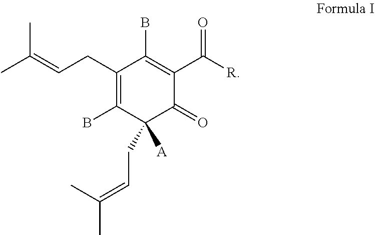

Hops Alpha Acids. A is the acidic hydroxyl functional group in the alpha position, B are the acidic hydroxyl functional groups in the beta position, and R is an alkyl functional group.

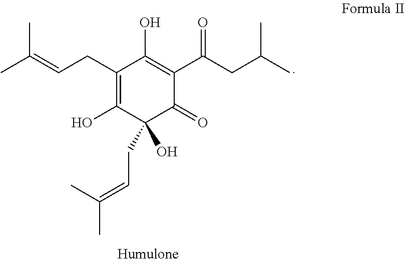 [SMILES: CC(C)=CCC1=C(O)[C@@](O)(CC=C(C)C)C(=O)C(C(=O)CC(C)C)=C1O ; "."]

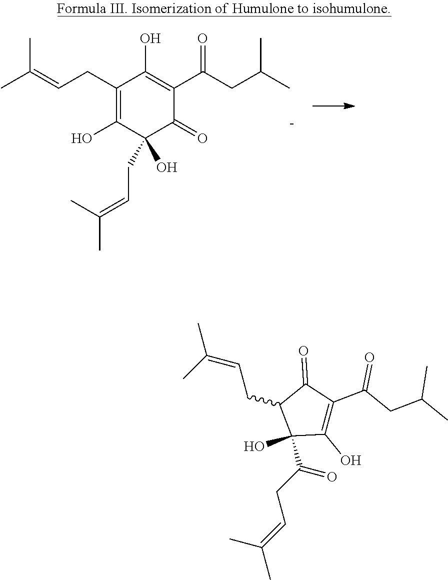

Suitable hops beta acids can include lupulone, adlupulone, colupulone, and/or mixtures thereof. A suitable hops beta acid can include a compound a described in Formula IV, V, VI, and/or VII.

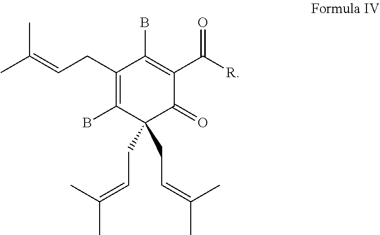

Hops Beta Acids. B are the acidic hydroxyl functional groups in the beta position and R is an alkyl functional group.

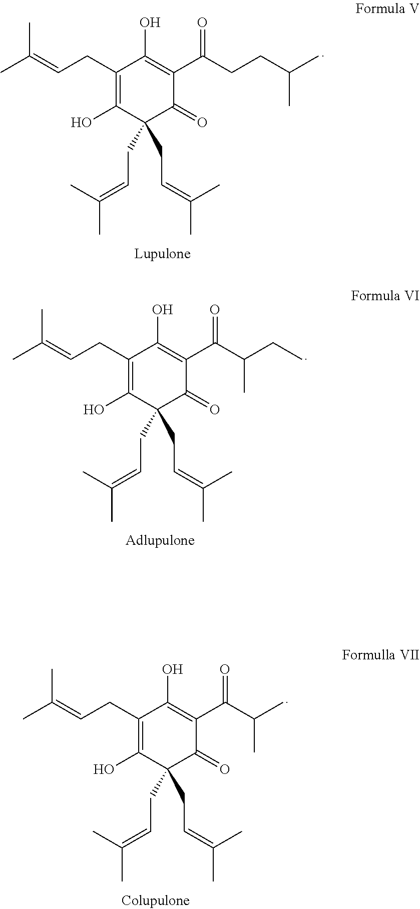

While hops alpha acids can demonstrate some antibacterial activity, hops alpha acids also have a bitter taste. The bitterness provided by hops alpha acids can be suitable for beer, but they are not suitable for use in oral care compositions. In contrast, hops beta acids can be associated with a higher antibacterial and/or anticaries activity, but not as bitter a taste. Thus, a hops extract with a higher proportion of beta acids to alpha acids than normally found in nature, can be suitable for use in oral care compositions for use as an antibacterial and/or anticaries agent.

A natural hops source can comprise from about 2% to about 12%, by weight of the hops source, of hops beta acids depending on the variety of hops. Hops extracts used in other contexts, such as in the brewing of beer, can comprise from about 15% to about 35%, by weight of the extract, of hops beta acids. The hops extract desired herein can comprise at least about 35%, at least about 40%, at least about 45%, from about 35% to about 95%, from about 40% to about 90%, or from about 45% to about 99%, of hops beta acids. The hops beta acids can be in an acidic form (i.e. with attached hydrogen atom(s) to the hydroxyl functional group(s)) or as a salt form.

A suitable hops extract is described in detail in U.S. Pat. No. 7,910,140, which is herein incorporated by reference in its entirety. The hops beta acids desired can be non-hydrogenated, partially hydrogenated by a non-naturally occurring chemical reaction, or hydrogenated by a non-naturally occurring chemical reaction. The hops beta acid can be essentially free of or substantially free of hydrogenated hops beta acid and/or hops acid. A non-naturally occurring chemical reaction is a chemical reaction that was conducted with the aid of chemical compound not found within Humulus lupulus, such as a chemical hydrogenation reaction conducted with high heat not normally experienced by Humulus lupulus in the wild and/or a metal catalyst.

A natural hops source can comprise from about 2% to about 12%, by weight of the hops source, of hops alpha acids. Hops extracts used in other contexts, such as in the brewing of beer, can comprise from about 15% to about 35%, by weight of the extract, of hops alpha acids. The hops extract desired herein can comprise less than about 10%, less than about 5%, less than about 1%, or less than about 0.5%, by weight of the extract, of hops alpha acids.

Hops oils can include terpene hydrocarbons, such as myrcene, humulene, caryophyllene, and/or mixtures thereof. The hops extract desired herein can comprise less than 5%, less than 2.5%, or less than 2%, by weight of the extract, of one or more hops oils.

Flavonoids present in the hops extract can include xanthohumol, 8-prenylnaringenin, isoxanthohumol, and/or mixtures thereof. The hops extract can be substantially free of, essentially free of, free of, or have less than 250 ppm, less than 150 ppm, and/or less than 100 ppm of one or more flavonoids.

As described in U.S. Pat. No. 5,370,863, hops acids have been previously added to oral care compositions. However, the oral care compositions taught by U.S. Pat. No. 5,370,863 only included up to 0.01%, by weight of the oral care composition. While not wishing to be bound by theory, it is believed that U.S. Pat. No. 5,370,863 could only incorporate a low amount of hops acids because of the bitterness of hops alpha acids. A hops extract with a low level of hops alpha acids would not have this concern.

The hops compound can be combined with or free from an extract from another plant, such as a species from genus Magnolia. The hops compounds can be combined with or free from triclosan.

The oral care composition can comprise from about 0.01% to about 10%, greater than 0.01% to about 10%, from about 0.05%, to about 10%, from about 0.1% to about 10%, from about 0.2% to about 10%, from about 0.2% to about 10%, from about 0.2% to about 5%, from about 0.25% to about 2%, from about 0.05% to about 2%, or from greater than 0.25% to about 2%, of hops, such as hops beta acid, as described herein. The hops, such as the hops beta acid, can be provided by a suitable hops extract, the hops plant itself, or a synthetically derived compound. The hops, such as hops beta acid, can be provided as neutral, acidic compounds, and/or as salts with a suitable counter ion, such as sodium, potassium, ammonia, or any other suitable counter ion.

The hops can be provided by a hops extract, such as an extract from Humulus lupulus with at least 35%, by weight of the extract, of hops beta acid and less than 1%, by weight of the hops extract, of hops alpha acid. The oral care composition can comprise 0.01% to about 10%, greater than 0.01% to about 10%, from about 0.05%, to about 10%, from about 0.1% to about 10%, from about 0.2% to about 10%, from about 0.2% to about 10%, from about 0.2% to about 5%, from about 0.25% to about 2%, from about 0.05% to about 2%, or from greater than 0.25% to about 2%, of hops extract, as described herein.

### Polyphosphate

The oral care composition can comprise polyphosphate, which can be provided by a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred polyphosphates include linear polyphosphates having the formula: XO(XPO₃)ₙX, wherein X is sodium, potassium, ammonium, or any other alkali metal cations and n averages from about 2 to about 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of, essentially free of, or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 20%, or about 10% or less, by weight of the oral care composition, of the polyphosphate source. Alternatively, the oral care composition can be essentially free of, substantially free of, or free of polyphosphate

### Whitening Agent

The oral care composition may comprise from about 0.1% to about 10%, from about 0.2% to about 5%, from about 1% to about 5%, or from about 1% to about 15%, by weight of the oral care composition, of a whitening agent. The whitening agent can be a compound suitable for whitening at least one tooth in the oral cavity. The whitening agent may include peroxides, metal chlorites, perborates, percarbonates, peroxyacids, persulfates, dicarboxylic acids, and combinations thereof. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, pthalamidoperoxycaproic acid, or mixtures thereof.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Oral Care Composition Forms

Suitable compositions forms include emulsion compositions, such as the emulsions compositions of U.S. Pat. No. 11,147,753, which is herein incorporated by reference in its entirety, unit-dose compositions, such as the unit-dose compositions of U.S. Patent Application Publication No. 2019/0343732, which is herein incorporated by reference in its entirety, leave-on oral care compositions, jammed emulsions, such as the jammed oil-in-water emulsions of U.S. Pat. No. 11,096,874, which is herein incorporated by reference in its entirety, dentifrice compositions, mouth rinse compositions, mouthwash compositions, tooth gel, subgingival gel, mouth rinse, mousse, foam, mouth spray, lozenge, chewable tablet, chewing gum, tooth whitening strips, floss and floss coatings, breath freshening dissolvable strips, denture care products, denture adhesive products, or combinations thereof.

### Methods

The oral care compositions, as described herein, can lead to oral health benefits, such as the treatment, reduction, and/or prevention of caries, cavities, gingivitis, and/or combinations thereof and/or the whitening of teeth, removing stain from teeth, and/or preventing the accumulation of stain from teeth when applied to the oral cavity. For example, a user can dispense at least a one-inch strip of a suitable oral care composition, as described herein, onto an oral care implement, such as a toothbrush, applicator, and/or tray, and applied to the oral cavity and/or teeth.

The user can be instructed to brush teeth thoroughly for at least 30 seconds, at least one minute, at least 90 seconds, or at least two minutes at least once, at least twice, or at least three times per day. The user can also be instructed to expectorate the oral care composition after the completion of the brush procedure.

The user can also be instructed to rinse with a mouthwash and/or mouth rinse composition after the completion of the brush procedure or instead of the brush procedure. The user can be instructed to swish the oral care composition thoroughly for at least 30 seconds, at least one minute, at least 90 seconds, or at least two minutes at least once, at least twice, or at least three times per day. The user can also be instructed to expectorate the oral care composition after the completion of the procedure.

The oral care compositions according to embodiments of the present invention can be used in the treatment, reduction, and/or prevention of caries, cavities, gingivitis, and/or combinations thereof. The oral care compositions according to embodiments of the present invention can be used to provide a whitening benefit, such as the whitening of teeth, removing stain from teeth, and/or preventing the accumulation of stain from teeth. For example, as described herein, hops beta acid can be useful as an antigingivitis agent. Thus, the addition of hops to any oral care composition can provide antigingivitis protection.

The oral care composition can include primary packaging, such as a tube, bottle, and/or tub. The primary package can be placed within secondary package, such as a carton, shrink wrap, or the like. Instructions for use of the oral care composition can be printed on the primary package and/or the secondary package. The scope of the method is intended to include instructions provided by a manufacturer, distributor, and/or producer of the oral care composition.

If the oral care composition is a toothpaste, the user can be instructed to dispense the toothpaste from the toothpaste tube.

The user can be instructed to apply a portion of the toothpaste onto a toothbrush. The portion of the toothpaste can be of any suitable shape, such as strip, a pea-sized amount, or various other shapes that would fit onto any mechanical and/or manual brush head. The user can be instructed to apply a strip of the toothpaste that is at least about 1 inch, at least about 0.5 inch, at least 1 inch, and/or at least 0.5 inch long to the bristles of a toothbrush, such as soft-bristled toothbrush.

The user can be instructed to apply pea-sized or grain of rice-sized portion of the toothpaste to the bristles of a toothbrush, such as in the case of use by children of less than 6 years old and/or less than 2 years old. The user can be instructed to brush their teeth for at least about 30 seconds, at least about 1 minute, at least about 90 seconds, at least about 2 minutes, at least 30 seconds, at least 1 minute, at least 90 seconds, and/or at least 2 minutes. The user can be instructed to brush their teeth thoroughly and/or as directed by a physician and/or dentist.

The user can be instructed to brush their teeth after each meal. The user can be instructed to brush their teeth at least once per day, at least twice per day, and/or at least three times per day. The user can be instructed to brush their teeth no more than three times a day, such as to prevent Sn staining. The user can be instructed to brush their teeth in the morning and/or in the evening prior to sleeping.

The user can be instructed to not swallow the toothpaste composition due to the inclusion of ingredients that are not suitable for ingestion, such as fluoride. The user may be instructed to expectorate (or spit out) the toothpaste composition after the cessation of the brushing cycle.

If the oral care composition is a mouth rinse, the user can be instructed to dispense the mouth rinse from a bottle containing the mouth rinse. The user can be instructed to use the mouth rinse at least once a day, at least twice a day, and/or at least three times a day. The user can be instructed to use the mouth rinse composition after the use of toothpaste and/or floss. The user can be instructed to swish a portion of rinse in the oral cavity, such as between the teeth, for a period of time. The user can be instructed to vigorously swish a portion of the rinse. The user can be instructed to use be from about 5 mL to about 50 mL, from about 10 mL to about 40 mL, 10 mL, 20 mL, 25 mL, 30 mL, 40 mL, 2 teaspoonfuls, and/or 4 teaspoonfuls of mouth rinse. The user can be instructed to swish the mouth rinse for at least about 30 seconds, at least about 1 minute, at least about 90 seconds, at least about 2 minutes, at least 30 seconds, at least 1 minute, at least 90 seconds, and/or at least 2 minutes. The user can be instructed to not swallow the mouth rinse composition due to the inclusion of ingredients that are not suitable for ingestion, such as fluoride. However, in the case of an oral care composition comprising hops, but free of fluoride, the user may not need to be instructed to not swallow the mouth rinse. The user may be instructed to expectorate (or spit out) the mouth rinse composition after the cessation of the rinse cycle.

The usage instructions for the oral care composition, such as for a toothpaste composition and/or a mouth rinse composition, can vary based on age. For example, adults and children that are at least 6 or at least 2 can have one usage instruction while children under 6 or under 2 can have a second usage instruction.

The oral care composition, as described herein, can be useful as medicament, such as in an anticavity and/or antigingivitis treatment, as described herein. Suitable medicaments include oral care compositions, toothpaste compositions, mouth rinse compositions, floss coatings, chewing gums, and/or other suitable compositions to be applied in the oral cavity.

Additionally, the oral care composition, as described herein, can be used to reduce the number and/or intensity of white spots on teeth, which can be attributable to caries presence within the oral cavity. Or the oral care composition, as described herein, can be used to reduce the redness, puffiness, tenderness, and/or swollenness of gums at the gumline immediately adjacent the surfaces of the teeth, which can be attributable to gingivitis presence within the oral cavity.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

### Fluoride Uptake

The Enamel Fluoride Uptake by FDA Method #40 is a method used to determine the amount of fluoride delivered to demineralized enamel specimens from a single, 30-minute treatment of 1:3 dentifrice slurry supernatant.

Cores of sound human enamel with diameters of 3-4 mm were extracted from whole human teeth. The cores were mounted on acrylic rods and the surfaces were ground using 600 grit wet/dry sandpaper. The cores were then polished with 0.05μ polish (Alumina Suspension Gamma B, MetLab Corp, catalog #M303-128) to a mirror finish. Specimens were stored in an airtight container above a small amount of deionized water (˜1-5 mL) in a standard laboratory refrigerator (˜2-4° C.).

Each enamel specimen was inspected and samples with large cracks or uneven calcification were discarded. Specimens were polished again for 10 minutes using 0.05μ polish. Samples were sonicated with a sonicator in deionized water for 15-30 min. Enamel specimens were then rinsed with standard deionized water and wiped to remove any residual polish.

Enamel specimens were then demineralized. For each specimen, 25 mL of MHDP (N-2-hydroxyethyl, methane hydroxy diphosphonate) demineralization solution (0.025M lactic acid, 2×10⁻⁴M MHDP) was placed in a 30 mL plastic vial. An enamel specimen was placed through the cap of each vial. Each cap was placed on the top of the vial to submerge the enamel specimen in the MHDP demineralization solution. The enamel specimen was not allowed to touch the bottom of the vial. Specimens were left in the demineralization solution for 48 hours at ambient conditions to form artificial caries lesions. The rods were tapped twice daily to remove any bubbles. After 48 hours, specimens were removed from the demineralization solution and rinsed thoroughly with deionized water.

If the sample was a paste dentifrice, 10 g of dentifrice was placed in a 50 mL tri-pour plastic beaker. 30 mL of deionized water was added to the beaker. An x-shaped stir bar was placed on top of the dentifrice in each beaker and the beaker was placed on a magnetic stir plate. The dentifrice was broken up with a wooden stick until the stir bar was capable of spinning freely at 300-400 rpm. The dentifrice slurry was stirred for 20 minutes. The slurry was transferred to a centrifuge tube and centrifuged for 30 minutes at 11,000 rpm.

Slurry supernatants were decanted into a 50 mL tri-pour plastic beaker. An x-shaped stir bar was placed in the beaker and the beaker was placed on a magnetic stir plate. The stir plate was turned to 300-400 rpm. Lesioned enamel specimens were suspended into each treatment. Each sample was treated for 30 minutes. After 30 minutes, each sample was rinsed with deionized water. Samples were stored in an airtight container above a small amount of deionized water (˜1-5 mL) in a standard laboratory refrigerator (˜2-4° C.).

The samples were analyzed for fluoride content analysis by collecting a portion of milled enamel powder following drilling to a depth of 50 micro-meters, dissolving that enamel in acid, then neutralizing and buffering it. Upon drilling a sample from the enamel specimen, the area of the enamel drilled was recorded.

Fluoride uptake was directly measured using a Fluoride Ion Specific Electrode (Thermo Scientific, Orion, 96-09-00, Waltham, MA, USA). Each specimen sample was placed on the end of the electrode. A value of mV was recorded. This value was converted to ppm fluoride by using a standard curve of prepared fluoride standards. Fluoride uptake was calculated by dividing the mass of fluoride in μg by the total area sampled with the microdrill biopsy.

### HAP Dissolution

The HAP dissolution method was designed to test the acid protection of a chosen test oral care composition. After treating hydroxyapatite powder (HAP) with test diluted solutions, the HAP was added to an acidic media and the change in pH was an indicator of the degree of surface protection from acid.

Diluted solutions (1:3 concentrate solution:water) were prepared for all treatment compositions. Specifically, 10 g of oral care composition was combined with 30 g of deionized, ultra-pure water in a 50 mL container with a stir bar. If a dentifrice was used, 10 g the dentifrice was broken up with a spatula until the stir bar moved freely at 300-400 rpm. The slurry was mixed on the stir plate for 10-20 minutes and/or until a uniform slurry was formed. The paste slurries were centrifuged at 15,000 rpm for 15 min to separate the solid components from the supernatant. If solutions were used, they were used without this centrifugation step.

For each treatment, including for the water control, 0.300 g of hydroxyapatite powder (HAP) was placed into a 50 mL round bottom centrifuge tube with 4, 4 mm glass beads. For treatment with an oral care composition, 24 mL of the prepared dentifrice supernatant or diluted solution was added to the HAP. Each treated HAP sample was immediately vortex mixed at 2500 rpm for 2 minutes. All samples were then centrifuged at 15,000 rpm for 15 minutes. The liquid phase was decanted out of the centrifuge tube, which left a HAP pellet and glass beads. The remaining HAP pellet was rinsed by adding deionized water, vortex mixing at 2500 rpm for 1 minute to completely disperse the pellet, centrifuging at 15,000 rpm for 15 minutes, and the liquid phase was decanted out of the centrifuge tube then discarded. This rinsing step was repeated two more times. The treated HAP pellet was dried in a 55° C. oven overnight.

Samples of HAP were analyzed for ΔpH. 25 mL of 10 mM citric acid (1.9212 g of citric acid in 1 L of deionized water) was added to a 50 mL beaker with a stir bar. The beaker was placed on a stir plate (Metrohm, Herisau, Switzerland, Model No. 728) and turned on. The Titrano pH electrode (Metrohm, Herisau, Switzerland, Model No. 719S) was placed in the stirring beaker with citric acid. After equilibration of the citric acid solution (until pH reads of 2.5±0.001 pH for 30 seconds), 50 mg of the dried HAP powder was added to the citric acid solution. The pH was monitored and the value at 5 min was recorded. The ΔpH is determined by subtracting the pH reading at 5 minutes from the stable pH reading obtained immediately prior to adding the treated HAP powder. The results for HAP solubility reduction are provided herein.

Example oral care compositions were made by dissolving the necessarly carboxylic acid(s) in 90 mL of ultra-pure water in a glass beaker using a stir bar and magnetic stir plate. After the acid(s) were completely dissolved, the stannous fluoride (0.454 g per 100 g solution) was added and stirred until completely dissolved. The solution was then neutralized dropwise with 1N NaOH dropwise until pH 4.5 was reached. The solution was transferred to a 100 mL Erlenmeyer flask and ultra-pure water was added to reach 100 mL. The solutions where then tightly stoppered and stored in a refrigerator until use. To treat the HAP powder, a quantity of solution was diluted 1 part solution (10 g) and 3 parts ultra-pure water (30 g) as if it were a toothpaste. The HAP powder was then treated as normal according to the method above.

The amount of chelate stabilizer used was defined by the molar ratio as indicated in TABLE 1-TABLE 8. By way of an illustrative example, Ex. 1 has one mol of gluconate ion for every 1 mol of stannous ion when the stannous in the composition is obtained by adding 0.454 g stannous fluoride per 100 g of solution. In this way, Ex. 1 contains 0.454 g stannous fluoride per 100 g of solution and 0.568 g gluconic acid per 100 g of solution.

By way of a second illustrative example, Ex. 56 has one mol of malonate and 1.67 mols of oxalate for every one mol of stannous. In this way, Ex. 56 contains 0.454 g stannous fluoride per 100 g solution, 0.302 g malonic acid per 100 g solution, and 0.436 g oxalic acid per 100 g solution.

TABLE 1
Molar Ratios of Sn to Stabilizers (Sn:Stabilizer) in Solution (0.454% SnF₂)

| | Gluconate | Lactate | Malonate | Oxalate | Malate | Tartrate | Citrate | Total |
|---|---|---|---|---|---|---|---|---|
| Ex. 1 | 1:1 | 0 | 0 | 0 | 0 | 0 | 0 | 1:1 |
| Ex. 2 | 1:2 | 0 | 0 | 0 | 0 | 0 | 0 | 1:2 |
| Ex. 3 | 1:3 | 0 | 0 | 0 | 0 | 0 | 0 | 1:3 |
| Ex. 4 | 0 | 1:1 | 0 | 0 | 0 | 0 | 0 | 1:1 |
| Ex. 5 | 0 | 1:2 | 0 | 0 | 0 | 0 | 0 | 1:2 |
| Ex. 6 | 0 | 1:3 | 0 | 0 | 0 | 0 | 0 | 1:3 |
| Ex. 7 | 0 | 0 | 1:1 | 0 | 0 | 0 | 0 | 1:1 |
| Ex. 8 | 0 | 0 | 1:2 | 0 | 0 | 0 | 0 | 1:2 |
| Ex. 9 | 0 | 0 | 1:3 | 0 | 0 | 0 | 0 | 1:3 |
| Ex. 10 | 0 | 0 | 0 | 1:1 | 0 | 0 | 0 | 1:1 |
| Ex. 11 | 0 | 0 | 0 | 1:2 | 0 | 0 | 0 | 1:2 |
| Ex. 12 | 0 | 0 | 0 | 1:3 | 0 | 0 | 0 | 1:3 |
| Ex. 13 | 0 | 0 | 0 | 0 | 1:1 | 0 | 0 | 1:1 |
| Ex. 14 | 0 | 0 | 0 | 0 | 1:2 | 0 | 0 | 1:2 |
| Ex. 15 | 0 | 0 | 0 | 0 | 1:3 | 0 | 0 | 1:3 |
| Ex. 16 | 0 | 0 | 0 | 0 | 0 | 1:1 | 0 | 1:1 |
| Ex. 17 | 0 | 0 | 0 | 0 | 0 | 1:2 | 0 | 1:2 |
| Ex. 18 | 0 | 0 | 0 | 0 | 0 | 1:3 | 0 | 1:3 |

TABLE 2
Molar Ratios of Sn to Stabilizers (Sn:Stabilizer) in Solution (0.454% SnF₂)

| | Gluconate | Lactate | Malonate | Oxalate | Malate | Tartrate | Citrate | Total |
|---|---|---|---|---|---|---|---|---|
| Ex. 19 | 1:1 | 1:1 | 0 | 0 | 0 | 0 | 0 | 1:2 |
| Ex. 20 | 1:1 | 0 | 1:1 | 0 | 0 | 0 | 0 | 1:2 |
| Ex. 21 | 1:1 | 0 | 0 | 1:1 | 0 | 0 | 0 | 1:2 |
| Ex. 22 | 1:1 | 0 | 0 | 0 | 1:1 | 0 | 0 | 1:2 |
| Ex. 23 | 1:1 | 0 | 0 | 0 | 0 | 1:1 | 0 | 1:2 |
| Ex. 24 | 0 | 1:1 | 1:1 | 0 | 0 | 0 | 0 | 1:2 |
| Ex. 25 | 0 | 1:1 | 0 | 1:1 | 0 | 0 | 0 | 1:2 |
| Ex. 26 | 0 | 1:1 | 0 | 0 | 1:1 | 0 | 0 | 1:2 |
| Ex. 27 | 0 | 1:1 | 0 | 0 | 0 | 1:1 | 0 | 1:2 |
| Ex. 28 | 1:1 | 1:2 | 0 | 0 | 0 | 0 | 0 | 1:3 |
| Ex. 29 | 1:1 | 0 | 1:2 | 0 | 0 | 0 | 0 | 1:3 |
| Ex. 30 | 1:1 | 0 | 0 | 1:2 | 0 | 0 | 0 | 1:3 |
| Ex. 31 | 1:1 | 0 | 0 | 0 | 1:2 | 0 | 0 | 1:3 |
| Ex. 32 | 1:1 | 0 | 0 | 0 | 0 | 1:2 | 0 | 1:3 |
| Ex. 33 | 0 | 1:1 | 1:2 | 0 | 0 | 0 | 0 | 1:3 |
| Ex. 34 | 0 | 1:1 | 0 | 1:2 | 0 | 0 | 0 | 1:3 |
| Ex. 35 | 0 | 1:1 | 0 | 0 | 1:2 | 0 | 0 | 1:3 |
| Ex. 36 | 0 | 1:1 | 0 | 0 | 0 | 1:2 | 0 | 1:3 |

TABLE 3
Molar Ratios of Sn to Stabilizers (Sn:Stabilizer) in Solution (0.454% SnF₂)

| | Gluconate | Lactate | Malonate | Oxalate | Malate | Tartrate | Citrate | Total |
|---|---|---|---|---|---|---|---|---|
| Ex. 37 | 1:2 | 1:1 | 0 | 0 | 0 | 0 | 0 | 1:3 |
| Ex. 38 | 1:2 | 0 | 1:1 | 0 | 0 | 0 | 0 | 1:3 |
| Ex. 39 | 1:2 | 0 | 0 | 1:1 | 0 | 0 | 0 | 1:3 |
| Ex. 40 | 1:2 | 0 | 0 | 0 | 1:1 | 0 | 0 | 1:3 |
| Ex. 41 | 1:2 | 0 | 0 | 0 | 0 | 1:1 | 0 | 1:3 |
| Ex. 42 | 0 | 1:2 | 1:1 | 0 | 0 | 0 | 0 | 1:3 |
| Ex. 43 | 0 | 1:2 | 0 | 1:1 | 0 | 0 | 0 | 1:3 |
| Ex. 44 | 0 | 1:2 | 0 | 0 | 1:1 | 0 | 0 | 1:3 |
| Ex. 45 | 0 | 1:2 | 0 | 0 | 0 | 1:1 | 0 | 1:3 |
| Ex. 46 | 0 | 0 | 1:1 | 1:1 | 0 | 0 | 0 | 1:2 |
| Ex. 47 | 0 | 0 | 1:1 | 1:2 | 0 | 0 | 0 | 1:3 |
| Ex. 48 | 0 | 0 | 1:2 | 1:1 | 0 | 0 | 0 | 1:3 |

TABLE 4
Molar Ratios of Sn to Stabilizers (Sn:Stabilizer) in Solution (0.454% SnF₂)

| | Gluconate | Lactate | Malonate | Oxalate | Malate | Tartrate | Citrate | Total |
|---|---|---|---|---|---|---|---|---|
| Ex. 49 | 1:1 | 0 | 0 | 0 | 0 | 0 | 1:1 | 1:2 |
| Ex. 50 | 0 | 0 | 1:1 | 0 | 0 | 0 | 1:1 | 1:2 |
| Ex. 51 | 0 | 0 | 0 | 1:1 | 0 | 0 | 1:1 | 1:2 |
| Ex. 52 | 0 | 0 | 0 | 0 | 1:1 | 0 | 1:1 | 1:2 |
| Ex. 53 | 0 | 0 | 0 | 0 | 0 | 1:1 | 1:1 | 1:2 |

TABLE 5
Molar Ratios of Sn to Stabilizers (Sn:Stabilizer) in Solution (0.454% SnF₂)

| | Gluconate | Lactate | Malonate | Oxalate | Malate | Tartrate | Citrate | Total |
|---|---|---|---|---|---|---|---|---|
| Ex. 54 | 0 | 0 | 1:1 | 1:1 | 0 | 0 | 0 | 1:2 |
| Ex. 55 | 0 | 0 | 1:1 | 1:1.33 | 0 | 0 | 0 | 1:2.33 |
| Ex. 56 | 0 | 0 | 1:1 | 1:1.67 | 0 | 0 | 0 | 1:2.67 |
| Ex. 57 | 0 | 0 | 1:1 | 1:2 | 0 | 0 | 0 | 1:3 |
| Ex. 58 | 0 | 0 | 1:1.33 | 1:2 | 0 | 0 | 0 | 1:3.33 |
| Ex. 59 | 0 | 0 | 1:1.67 | 1:2 | 0 | 0 | 0 | 1:3.67 |
| Ex. 60 | 0 | 0 | 1:2 | 1:2 | 0 | 0 | 0 | 1:4 |

TABLE 6
Molar Ratios of Sn to Stabilizers (Sn:Stabilizer) in Solution (0.454% SnF₂)

| | Gluconate | Lactate | Malonate | Oxalate | Malate | Tartrate | Citrate | Total |
|---|---|---|---|---|---|---|---|---|
| Ex. 61 | 1:1 | 0 | 0 | 0 | 0 | 0 | 1:1 | 1:2 |
| Ex. 62 | 1:0.75 | 0 | 0 | 1:0.5 | 0 | 0 | 1:0.75 | 1:2 |
| Ex. 63 | 1:0.5 | 0 | 0 | 1:1 | 0 | 0 | 1:0.5 | 1:2 |
| Ex. 64 | 1:0.25 | 0 | 0 | 1:1.5 | 0 | 0 | 1:0.25 | 1:2 |
| Ex. 65 | 0 | 0 | 0 | 1:2 | 0 | 0 | 0 | 1:2 |
| Ex. 66 | 1:1 | 0 | 0 | 1:1 | 0 | 0 | 1:1 | 1:3 |
| Ex. 67 | 1:0.75 | 0 | 0 | 1:2 | 0 | 0 | 1:1 | 1:4 |

TABLE 7
Molar Ratios of Sn to Stabilizers (Sn:Stabilizer) in Solution (0.454% SnF₂)

| | Gluconate | Lactate | Malonate | Oxalate | Malate | Tartrate | Citrate | Total |
|---|---|---|---|---|---|---|---|---|
| Ex. 68 | 0 | 1:1 | 0 | 0 | 0 | 0 | 1:1 | 1:2 |
| Ex. 69 | 0 | 1:0.75 | 0 | 1:0.5 | 0 | 0 | 1:0.75 | 1:2 |
| Ex. 70 | 0 | 1:0.5 | 0 | 1:1 | 0 | 0 | 1:0.5 | 1:2 |
| Ex. 71 | 0 | 1:0.25 | 0 | 1:1.5 | 0 | 0 | 1:0.25 | 1:2 |
| Ex. 72 | 0 | 0 | 0 | 1:2 | 0 | 0 | 0 | 1:2 |
| Ex. 73 | 0 | 1:1 | 0 | 1:1 | 0 | 0 | 1:1 | 1:3 |
| Ex. 74 | 0 | 1:0.75 | 0 | 1:2 | 0 | 0 | 1:1 | 1:4 |

TABLE 8
Molar Ratios of Sn to Stabilizer (Sn:Stabilizer)
in Solution (0.454% SnF₂)

| | Disodium EDTA | Total |
|---|---|---|
| Ex. 75 | 1:1 | 1:1 |

TABLE 9
Molar Ratios of Sn to Stabilizer (Sn:Stabilizer) in Solution (0.454% SnF₂)

| | Gluconate | Lactate | Malonate | Oxalate | Malate | Tartrate | Citrate | Total |
|---|---|---|---|---|---|---|---|---|
| Ex. 76 | 1:1 | 0 | 0 | 0 | 0 | 0 | 1:1 | 1:2 |

The molar ratios in Ex. 76 are identical to that of Ex. 49; however, the target pH of the composition in TABLE 9 is 7.

The results for HAP Solubility Reduction by the HAP Dissolution method; Enamel Fluoride Uptake by the Fluoride Uptake method; and the treatment pH for the inventive examples are described below. The values for the inventive examples are compared to those obtained for sodium fluoride in water at pH 4.5, Crest Cavity Protection, USP Sodium Fluoride Reference Dentifrice, ultra-pure water, and a 1:1:1 mixture of stannous fluoride with gluconic acid and citric acid (Ex. 49).

TABLE A
Results for HAP Solubility Reduction, Enamel
Fluoride Uptake, and Fluoride Activity

| | HAP Solubility | Fluoride | |
|---|---|---|---|
| | Reduction | Uptake | |
| Treatment | (% vs. Water) | (μg F/cm²) | Treatment pH |
| Water | 0 | — | — |
| 0.243% NaF in Water | — | 20.93 | 4.5 |
| Crest Cavity Protection | 32.7 | — | 7.0 |
| USP Sodium Fluoride | — | 8.17 | 7.0 |
| Ex. 49 | 56.9* | 15.4\hat{ } | 4.5 |
| Ex. 1 | 76.5 | | 4.5 |
| Ex. 2 | 75.1 | | 4.5 |
| Ex. 3 | 75.2 | 8.24 | 4.5 |
| Ex. 4 | 76.3 | | 4.5 |
| Ex. 5 | 77.7 | | 4.5 |
| Ex. 6 | 76.4 | | 4.5 |
| Ex. 7 | 79.8 | | 4.5 |
| Ex. 8 | 74.1 | | 4.5 |
| Ex. 9 | 70.7 | 5.55 | 4.5 |
| Ex. 10 | 67.3* | | 4.5 |
| Ex. 11 | 62.0* | 19.9\hat{ } | 4.5 |
| Ex. 12 | 59.2* | 20.6\hat{ } | 4.5 |
| Ex. 13 | 72.7 | | 4.5 |
| Ex. 14 | 68.6* | | 4.5 |
| Ex. 15 | 65.9* | | 4.5 |
| Ex. 16 | 73.1 | | 4.5 |
| Ex. 17 | 66.8* | | 4.5 |
| Ex. 18 | 66.1* | | 4.5 |

The results in TABLE A illustrate the HAP Solubility Reduction and Fluoride Uptake performance of 1:1, 1:2, and 1:3 mixtures of stannous fluoride with gluconic acid (Ex. 1, 2, 3); lactic acid (Ex. 4, 5, 6); malonic acid (Ex. 7, 8, 9); oxalic acid (Ex. 10, 11, 12); malic acid (Ex. 13, 14, 15); and DL-tartaric acid (Ex. 16, 17, 18). The optimum HAP Solubility Reduction is between 35% and 70% reduction vs. water. Examples that achieve this optimum are indicated by “*”. The optimum Fluoride Uptake is greater than 10 μg F/cm². Examples that achieve this optimum are indicated by “\hat{ }”. Stannous fluoride stabilized by only gluconic, lactic, or malonic acid is unable to achieve an optimum for either HAP solubility reduction or fluoride uptake or any ratio of ligand to stannous. Oxalic acid in a 1:1, 1:2, or 1:3 molar ratio with stannous achieves an optimum HAP solubility reduction. Malic acid in a 1:2 or 1:3 molar ratio with stannous achieves an optimum HAP solubility reduction. DL-tartaric acid in a 1:2 or 1:3 molar ratio with stannous achieves an optimum HAP solubility reduction. Oxalic acid in a 1:2 or 1:3 molar ratio with stannous fluoride also achieved an optimum fluoride uptake.

TABLE B
Results for HAP Solubility Reduction, Enamel
Fluoride Uptake, and Fluoride Activity

| | HAP Solubility | Fluoride | |
|---|---|---|---|
| | Reduction | Uptake | |
| Treatment | (% vs. Water) | (μg F/cm²) | Target pH |
| Water | 0 | — | — |
| 0.243% NaF in Water | — | 20.93 | 4.5 |
| Crest Cavity Protection | | — | 7.0 |
| USP Sodium Fluoride | — | 8.17 | 7.0 |
| Ex. 49 | 56.9* | 15.4\hat{ } | 4.5 |
| Ex. 19 | 78.9 | | 4.5 |
| Ex. 20 | 80.4 | 7.8 | 4.5 |
| Ex. 21 | 64.7* | 10.6\hat{ } | 4.5 |
| Ex. 22 | 76.3 | | 4.5 |
| Ex. 23 | 72.9 | | 4.5 |
| Ex. 24 | 70.9 | | 4.5 |
| Ex. 25 | 63.8* | | 4.5 |
| Ex. 26 | 70.0* | | 4.5 |
| Ex. 27 | 65.1* | | 4.5 |
| Ex. 28 | 77.3 | | 4.5 |
| Ex. 29 | 76.4 | 7.5 | 4.5 |
| Ex. 30 | 60.0* | 20.5\hat{ } | 4.5 |
| Ex. 31 | 68.6* | | 4.5 |
| Ex. 32 | 66.9* | | 4.5 |
| Ex. 33 | 73.4 | | 4.5 |
| Ex. 34 | 58.0* | | 4.5 |
| Ex. 35 | 66.8* | | 4.5 |
| Ex. 36 | 63.8* | | 4.5 |

The results in TABLE B illustrate the HAP Solubility Reduction and Fluoride Uptake performance of 1:1:1 or 1:1:2 mixtures of stannous fluoride to ligand A to ligand B. The optimum HAP Solubility Reduction is between 35% and 70% reduction vs. water. Examples that achieve this optimum are indicated by “*”. The optimum Fluoride Uptake is greater than 10 μg F/cm². Examples that achieve this optimum are indicated by “\hat{ }”. With respect to HAP Solubility Reduction, only the 1:1:1 mixtures of stannous:gluconate:oxalate (Ex. 21); stannous:lactate:oxalate (Ex. 25); stannous:lactate:malate (Ex. 26); and stannous:lactate:DL-tartarate (Ex. 27) are able to achieve this optimum. Interestingly, more 1:1:1 mixtures work when lactate is used than when gluconate is used. For 1:1:2 mixtures, only the mixtures of stannous:gluconate:oxalate (Ex. 30); stannous:gluconate:malate (Ex. 31); stannous:gluconate:DL-tartarate (Ex. 32); stannous:lactate:oxalate (Ex. 34); stannous:lactate:malate (Ex. 35); and stannous:lacate:DL-tartarate (Ex. 36) achieve this optimum. Also interestingly, gluconate:malate and gluconate:DL-tartarate work at a 1:1:2 ratio and do not work at a 1:1:1 ratio. With respect to fluoride uptake a 1:1:1 stannous:gluconate:oxalate system (Ex. 21) delivered optimum fluoride uptake greater than 10 μg F/cm². However, when the ligand ratio was increased to 1:1:2 (Ex. 30), the fluoride uptake almost doubled.

TABLE C
Results for HAP Solubility Reduction, Enamel
Fluoride Uptake, and Fluoride Activity

| | HAP Solubility | Fluoride | |
|---|---|---|---|
| | Reduction | Uptake | |
| Treatment | (% vs. Water) | (μg F/cm²) | Target pH |
| Water | 0 | — | — |
| 0.243% NaF in Water | — | 20.93 | 4.5 |
| Crest Cavity Protection | 32.9 | — | 7.0 |
| USP Sodium Fluoride | — | 8.17 | 7.0 |
| Ex. 49 | 56.9* | 15.4\hat{ } | 4.5 |
| Ex. 37 | 74.5 | | 4.5 |
| Ex. 38 | 75.9 | 9.0 | 4.5 |
| Ex. 39 | 64.4* | 13.1\hat{ } | 4.5 |
| Ex. 40 | 72.0 | | 4.5 |
| Ex. 41 | 69.3* | | 4.5 |
| Ex. 42 | 75.1 | | 4.5 |
| Ex. 43 | 63.3* | | 4.5 |
| Ex. 44 | 71.7 | | 4.5 |
| Ex. 45 | 69.2* | | 4.5 |
| Ex. 46 | 66.4* | 8.6 | 4.5 |
| Ex. 47 | 60.1* | 17.8\hat{ } | 4.5 |
| Ex. 48 | 64.4* | 7.0 | 4.5 |

The results in TABLE C illustrate the HAP Solubility Reduction and Fluoride Uptake performance of 1:2:1 mixtures of stannous fluoride to ligand A to ligand B (Ex. 37-45) or mixtures of stannous with malonic acid and oxalic acid (Ex. 46-48). The optimum HAP Solubility Reduction is between 35% and 70% reduction vs. water. Examples that achieve this optimum are indicated by “*”. The optimum Fluoride Uptake is greater than 10 μg F/cm². Examples that achieve this optimum are indicated by “\hat{ }”. With respect to HAP Solubility Reduction, only the 1:2:1 mixtures of stannous:gluconate:oxalate (Ex. 39); stannous:gluconate:DL-tartarate (Ex. 41); stannous:lactate:oxalate (Ex. 43); and stannous:lactate:DL-tartarate (Ex. 45) are able to achieve this optimum. With respect to fluoride uptake a 1:2:1 stannous:gluconate:oxalate system (Ex. 39) delivered optimum fluoride uptake greater than 10 μg F/cm². In the case of mixtures of stannous with malonic acid and oxalic acid, a 1:1:1 mixture (Ex. 46) and a 1:2:1 mixture (Ex. 48) of stannous:malonate:oxalate achieved an optimum HAP solubility reduction but did not achieve an optimum fluoride uptake. Only a 1:1:2 mixture (Ex. 47) of stannous:malonate:oxalate achieved both an optimum solubility reduction and optimum fluoride uptake.

TABLE D
Results for HAP Solubility Reduction, Enamel
Fluoride Uptake, and Fluoride Activity

| | HAP Solubility | Fluoride | |
|---|---|---|---|
| | Reduction | Uptake | |
| Treatment | (% vs. Water) | (μg F/cm²) | Target pH |
| Water | 0 | — | — |
| 0.243% NaF in Water | — | 20.93 | 4.5 |
| Crest Cavity Protection | 29.7 | — | 7.0 |
| USP Sodium Fluoride | — | 8.17 | 7.0 |
| Ex. 49 | 56.9* | 15.4\hat{ } | 4.5 |
| Ex. 50 | 56.8* | 16.7\hat{ } | 4.5 |
| Ex. 51 | 56.0* | 14.0\hat{ } | 4.5 |
| Ex. 52 | 55.5* | | 4.5 |
| Ex. 53 | 54.3* | | 4.5 |

The results in TABLE D illustrate the HAP Solubility Reduction and Fluoride Uptake performance of 1:1:1 mixtures of stannous fluoride to didentate ligand to citric acid (Ex. 49-53). The results for Ex. 49 are a 1:1:1 mixture of stannous:gluconate:citrate. The optimum HAP Solubility Reduction is between 35% and 70% reduction vs. water. Examples that achieve this optimum are indicated by “*”. The optimum Fluoride Uptake is greater than 10 μg F/cm². Examples that achieve this optimum are indicated by “\hat{ }”. Ex. 50-53 all achieve an optimum HAP solubility reduction and fluoride uptake. Interestingly, the 1:1:1 mixtures of stannous:didentate:tridentate ligands appear to be especially effective at the stabilization of stannous to achieve both optimum solubility reduction and fluoride uptake.

TABLE E
Results for HAP Solubility Reduction, Enamel
Fluoride Uptake, and Fluoride Activity

| | HAP Solubility | Fluoride | |
|---|---|---|---|
| | Reduction | Uptake | |
| Treatment | (% vs. Water) | (μg F/cm²) | Target pH |
| Water | 0 | — | — |
| 0.243% NaF in Water | — | 20.93 | 4.5 |
| Crest Cavity Protection | 32.1 | — | 7.0 |
| USP Sodium Fluoride | — | 8.17 | 7.0 |
| Ex. 49 | 56.9* | 15.4\hat{ } | 4.5 |
| Ex. 54 | 63.2* | 8.2 | 4.5 |
| Ex. 55 | 62.2* | 10.9\hat{ } | 4.5 |
| Ex. 56 | 62.4* | 14.2\hat{ } | 4.5 |
| Ex. 57 | 59.0* | 17.9\hat{ } | 4.5 |
| Ex. 58 | 58.2* | 17.6\hat{ } | 4.5 |
| Ex. 59 | 55.6* | 17.4\hat{ } | 4.5 |
| Ex. 60 | 58.2* | 14.2\hat{ } | 4.5 |

The results in TABLE E illustrate the HAP Solubility Reduction and Fluoride Uptake performance of fractional mixtures of stannous fluoride to malonic acid to oxalic acid. Ex. 54-57 consider increasing the amount of oxalic acid while the malonic acid stays fixed increasing the ratio of ligands to stannous in the following order 1:1:1 (Ex. 54), 1:1:1.33 (Ex. 55); 1:1:1.67 (Ex. 56); and 1:1:2 (Ex. 57). Ex. 58-60 then increase the amount of malonic acid while the oxalic acid is fixed increasing the ratio of ligands to stannous in the following order 1:1.33:2 (Ex. 58); 1:1.67:2 (Ex. 59); and 1:2:2 (Ex. 60) The optimum HAP Solubility Reduction is between 35% and 70% reduction vs. water. Examples that achieve this optimum are indicated by “*”. The optimum Fluoride Uptake is greater than 10 μg F/cm². Examples that achieve this optimum are indicated by “\hat{ }”. Ex. 54-60 all achieve an optimum HAP solubility reduction. Only Ex. 54 fails to deliver optimum fluoride uptake with nearly identical results to Ex. 46. Interestingly, there appears to be an optimum fractional ratio of ligand to stabilizer for fluoride uptake from 1:1:2-1:1.33:2.

TABLE F
Results for HAP Solubility Reduction, Enamel
Fluoride Uptake, and Fluoride Activity

| | HAP Solubility | Fluoride | |
|---|---|---|---|
| | Reduction | Uptake | |
| Treatment | (% vs. Water) | (μg F/cm²) | Target pH |
| Water | 0 | — | — |
| 0.243% NaF in Water | — | 20.93 | 4.5 |
| Crest Cavity Protection | 32.1 | — | 7.0 |
| USP Sodium Fluoride | — | 8.17 | 7.0 |
| Ex. 49 | 56.9* | 15.4\hat{ } | 4.5 |
| Ex. 61 | 55.5* | 9.6\hat{ } | 4.5 |
| Ex. 62 | 57.1* | 10.8\hat{ } | 4.5 |
| Ex. 63 | 59.9* | 11.3\hat{ } | 4.5 |
| Ex. 64 | 59.5* | 15.0\hat{ } | 4.5 |
| Ex. 65 | 61.2* | 12.2\hat{ } | 4.5 |
| Ex. 66 | 53.3* | 13.1\hat{ } | 4.5 |
| Ex. 67 | 53.1* | 17.1\hat{ } | 4.5 |

The results in TABLE F illustrate the HAP Solubility Reduction and Fluoride Uptake performance of fractional mixtures of stannous fluoride to gluconic to oxalic to citric acid ligands. The results for Ex. 49 and Ex. 61 are a 1:1:1 mixture of stannous:gluconate:citrate. Differences in fluoride uptake represent the biological variability with respect to the Fluoride Uptake method. Ex. 62-65 consider increasing the amount of oxalic acid while decreasing the amount of gluconic and citric acid so that the molar sum of all of the ligands is 2× the molar amount of stannous. The ratio of gluconate:oxalate:citrate changes in the following order 0.75:0.5:0.75 (Ex. 62); 0.5:1:0.5 (Ex. 63); 0.25:1.5:0.25 (Ex. 64); and 0:2:0 (Ex. 65). All four examples achieve both optimal HAP solubility reduction and fluoride uptake. An apparent optimum fluoride uptake is achieved for Ex. 64. Ex. 66 and 67 increase the total stabilizer used. The ratio of gluconate:oxalate:citrate increases in the following order 1:1:1 (Ex. 66); and 1:2:1 (Ex. 67). Again, both examples achieve optimal HAP solubility reduction and fluoride uptake. An apparent optimum fluoride uptake is achieved for Ex. 67.

TABLE G
Results for HAP Solubility Reduction, Enamel
Fluoride Uptake, and Fluoride Activity

| | HAP Solubility | Fluoride | |
|---|---|---|---|
| | Reduction | Uptake | |
| Treatment | (% vs. Water) | (μg F/cm²) | Target pH |
| Water | 0 | — | — |
| 0.243% NaF in Water | — | 20.93 | 4.5 |
| Crest Cavity Protection | 32.1 | — | 7.0 |
| USP Sodium Fluoride | — | 8.17 | 7.0 |
| Ex. 49 | 56.9* | 15.4\hat{ } | 4.5 |
| Ex. 68 | 54.1* | 7.7 | 4.5 |
| Ex. 69 | 54.7* | 8.0 | 4.5 |
| Ex. 70 | 57.1* | 13.9\hat{ } | 4.5 |
| Ex. 71 | 57.4* | 10.4\hat{ } | 4.5 |
| Ex. 72 | 60.9* | 10.6\hat{ } | 4.5 |
| Ex. 73 | 54.4* | 14.3\hat{ } | 4.5 |
| Ex. 74 | 52.5* | 19.1\hat{ } | 4.5 |
| Ex. 75 | 40.8* | 23.0\hat{ } | 4.5 |

The results in TABLE G illustrate the HAP Solubility Reduction and Fluoride Uptake performance of fractional mixtures of stannous fluoride to lactic to oxalic to citric acid ligands. The results for Ex. 49 are a 1:1:1 mixture of stannous:gluconate:citrate. The results in TABLE G are for similar ratios of stabilizer as in TABLE F, but the gluconic acid has been swapped for lactic acid. Ex. 68-72 consider increasing the amount of oxalic acid while decreasing the amount of lactic and citric acid so that the molar sum of all of the ligands is 2× the molar amount of stannous. The ratio of lactate:oxalate:citrate changes in the following order 1:0:1 (Ex. 68); 0.75:0.5:0.75 (Ex. 69); 0.5:1:0.5 (Ex. 70); 0.25:1.5:0.25 (Ex. 71); and 0:2:0 (Ex. 72). Only Ex. 70-72 achieve both optimal HAP solubility reduction and fluoride uptake. An apparent optimum fluoride uptake is achieved for Ex. 70. Ex. 73 and 74 increase the total stabilizer used. The ratio of lactate:oxalate:citrate increases in the following order 1:1:1 (Ex. 73); and 1:2:1 (Ex. 74). Again, both examples achieve optimal HAP solubility reduction and fluoride uptake. An apparent optimum fluoride uptake is achieved for Ex. 74. Finally, Ex. 75 is for a stannous composition stabilized entirely by ethylenediaminetetraacetic acid (EDTA), which is a tetracarboxylic acid. Ex. 75 achieves both optimal HAP solubility reduction and fluoride uptake.

TABLE H
Results for HAP Solubility Reduction, Enamel
Fluoride Uptake, and Fluoride Activity

| | HAP Solubility | Fluoride | |
|---|---|---|---|
| | Reduction | Uptake | |
| Treatment | (% vs. Water) | (μg F/cm²) | Target pH |
| Water | 0 | — | — |
| Crest Cavity Protection | 32.1 | — | 7.0 |
| Ex. 76 | 54.9* | 8.0 | 7.0 |

The results in TABLE H illustrate the HAP Solubility Reduction and Fluoride Uptake performance of fractional mixtures of stannous fluoride to gluconic to citric acid ligands. The results for Ex. 76 are a 1:1:1 mixture of stannous:gluconate:citrate. For this composition, the optimum HAP solubility reduction was achieved; however, the optimum fluoride uptake was not. Without wishing to be bound by theory, we believe this is because the pH of the composition was not optimum for the combined amplification of HAP solubility reduction and enamel fluoride uptake.

The terms “substantially,” “essentially,” “about,” “approximately,” and the like, as may be used herein, represent the inherent degree of uncertainty that may be attributed to any quantitative comparison, value, measurement, or other representation. These terms also represent the degree by which a quantitative representation may vary from a stated reference without resulting in a change in the basic function of the subject matter at issue. Further, the dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition comprising:
a. an oral care active, wherein the oral care active comprises a fluoride ion source and a stannous ion source; and
b. a mixture of dentate ligands comprising:
i. a first polydentate ligand, wherein the first polydentate ligand is oxalic acid or a salt thereof, or a combination thereof; and
ii. a monodentate ligand, wherein the monodentate ligand is gluconic acid or a salt thereof, or a combination thereof, and a molar ratio of stannous ions to the mixture of dentate ligands is in a range of from about 1:1 to about 1:4; or
iii. a second polydentate ligand, wherein the second polydentate ligand comprises a tridentate ligand or a combination thereof, wherein the second polydentate ligand comprises a tricarboxylic acid, a salt thereof, or a combination thereof.

2. The oral care composition of claim 1, wherein the mixture of dentate comprises the monodentate ligand, and the molar ratio of the stannous ions to the mixture of dentate ligands is in a range of from about 1:1 to about 1:3.

3. The oral care composition of claim 1, wherein the mixture of dentate comprises the second polydentate ligand, and the tricarboxylic acid comprises citric acid, isocitric acid, aconitic acid, propane-1,2,3-tricarboxylic acid, trimesic acid, a salt thereof, or a combination thereof.

4. The oral care composition of claim 1, wherein the oral care composition comprises from about 1.5% to about 5.0%, by weight of the oral care composition, of the individual ligands.

5. The oral care composition of claim 1, wherein the fluoride ion source comprises stannous fluoride, sodium fluoride, sodium monofluorophosphate, amine fluoride, or combinations thereof.

6. The oral care composition of claim 1, wherein the composition comprises soluble fluoride ions of at least about 1000 ppm.

7. The oral care composition of claim 1, wherein the composition comprises from about 0.2% to about 1.0%, by weight of the oral care composition, of the stannous ion source, and wherein the stannous ion source comprises stannous fluoride, stannous chloride, or a combination thereof.

8. The oral care composition of claim 1, wherein a pH of the oral care composition is from about 4 to about 6.

9. An oral care composition comprising:
a. an oral care active, wherein the oral care active comprises a fluoride ion source and a stannous ion source; and
b. a mixture of dentate ligands comprising:
i. a first polydentate ligand, wherein the first polydentate ligand is oxalic acid or a salt thereof, or a combination thereof; and
ii. a monodentate ligand, wherein the monodentate ligand is gluconic acid or a salt thereof, or a combination thereof, and a molar ratio of stannous ions to the mixture of dentate ligands is in a range of from about 1:1 to about 1:4; and
iii. a second polydentate ligand, wherein the second polydentate ligand comprises a tridentate ligand or a combination thereof, wherein the second polydentate ligand comprises a tricarboxylic acid, a salt thereof, or a combination thereof.

10. The oral care composition of claim 9, wherein the molar ratio of the stannous ions to the mixture of dentate ligands is in a range of from about 1:1 to about 1:3.

11. The oral care composition of claim 9, wherein the tricarboxylic acid comprises citric acid, isocitric acid, aconitic acid, propane-1,2,3-tricarboxylic acid, trimesic acid, a salt thereof, or a combination thereof.

12. The oral care composition of claim 9, wherein the oral care composition comprises from about 1.5% to about 5.0%, by weight of the oral care composition, of the individual ligands.

13. The oral care composition of claim 9, wherein the fluoride ion source comprises stannous fluoride, sodium fluoride, sodium monofluorophosphate, amine fluoride, or combinations thereof, and wherein the composition comprises soluble fluoride ions of at least about 1000 ppm.

14. The oral care composition of claim 9, wherein the composition comprises from about 0.2% to about 1.0%, by weight of the oral care composition, of the stannous ion source, and wherein the stannous ion source comprises stannous fluoride, stannous chloride, or a combination thereof.

15. The oral care composition of claim 9, wherein a pH of the oral care composition is from about 4 to about 6.

16. An oral care composition comprising:
a. an oral care active, wherein the oral care active comprises a fluoride ion source and a stannous ion source; and
b. a polydentate ligand, wherein the polydentate ligand is a tetracarboxylic acid, wherein the tetracarboxylic acid comprises ethylenediaminetetraacetic acid, ethylenetetracarboxylic acid, ethane-1,1,2,2-tetracarboxylic acid, or a salt thereof, or a combination thereof; and
wherein the ratio of the stannous ions to the polydentate ligands is in the range of from about 1:1 to about 1:2.

17. The oral care composition of claim 16, wherein the oral care composition comprises from about 1.0% to about 7.5%, by weight of the oral care composition of the tetracarboxylic acid.

18. The oral care composition of claim 16, wherein the fluoride ion source comprises stannous fluoride, sodium fluoride, sodium monofluorophosphate, amine fluoride, or combinations thereof, and wherein the composition comprises soluble fluoride ions of at least about 1000 ppm.

19. The oral care composition of claim 16, wherein the composition comprises from about 0.2% to about 1.0% by weight of the oral care composition, of the stannous ion source, and wherein the stannous ion source comprises stannous fluoride, stannous chloride, or a combination thereof.

20. The oral care composition of claim 16, wherein a pH of the oral care composition is from about 4 to about 6.

21. A method for treating erosion, preventing erosion, treating caries, preventing caries, or a combination thereof, the method comprising:
a) depositing the oral care composition of claim 1 to a toothbrush;
b) applying the oral care composition to oral cavity surfaces by brushing;
c) letting the oral care composition acting on the oral cavity surfaces for at least 2 mins; and
d) removing the oral care composition from the oral cavity by expectorating and optionally rinsing.
